SEQUENCE LISTING (315 sequences)

SEQ ID NO: 1 - DNA, 21 bp, Artificial sequence
  Primer
aaaaaaagcaccgactcggtg

SEQ ID NO: 2 - DNA, 59 bp, Artificial sequence
  Primer
taatacgactcactataggtatcggatttatttatttaagttttagagctagaaatagc

SEQ ID NO: 3 - DNA, 59 bp, Artificial sequence
  Primer
taatacgactcactataggttatcggatttatttatttagttttagagctagaaatagc

SEQ ID NO: 4 - DNA, 59 bp, Artificial sequence
  Primer
taatacgactcactataggtttatcggatttatttattagttttagagctagaaatagc

SEQ ID NO: 5 - DNA, 59 bp, Artificial sequence
  Primer
taatacgactcactataggatttatcggatttatttattgttttagagctagaaatagc

SEQ ID NO: 6 - DNA, 59 bp, Artificial sequence
  Primer
taatacgactcactataggtatttatcggatttatttatgttttagagctagaaatagc

SEQ ID NO: 7 - DNA, 59 bp, Artificial sequence
  Primer
taatacgactcactataggttatttatcggatttatttagttttagagctagaaatagc

SEQ ID NO: 8 - DNA, 59 bp, Artificial sequence
  Primer
taatacgactcactataggattatttatcggatttatttgttttagagctagaaatagc

SEQ ID NO: 9 - DNA, 59 bp, Artificial sequence
  Primer
taatacgactcactataggtattatttatcggatttattgttttagagctagaaatagc

SEQ ID NO: 10 - DNA, 59 bp, Artificial sequence
  Primer
taatacgactcactataggattattattatcggatttatgttttagagctagaaatagc

SEQ ID NO: 11 - DNA, 59 bp, Artificial sequence
  Primer
taatacgactcactataggtattatatttatcggatttagttttagagctagaaatagc

SEQ ID NO: 12 - DNA, 59 bp, Artificial sequence
  Primer
taatacgactcactataggttattatatttatcggatttgttttagagctagaaatagc

SEQ ID NO: 13 - DNA, 59 bp, Artificial sequence
  Primer
taatacgactcactataggattattatatttatcggattgttttagagctagaaatagc

SEQ ID NO: 14 - DNA, 59 bp, Artificial sequence
  Primer
taatacgactcactataggtattattatatttatcggatgttttagagctagaaatagc

SEQ ID NO: 15 - DNA, 59 bp, Artificial sequence
  Primer
taatacgactcactataggattattattattatatcggagttttagagctagaaatagc

SEQ ID NO: 16 - DNA, 59 bp, Artificial sequence
  Primer
taatacgactcactataggattattattattattatatcgttttagagctagaaatagc

SEQ ID NO: 17 - DNA, 59 bp, Artificial sequence
  Primer
taatacgactcactataggtataggatttatttatttaagttttagagctagaaatagc

SEQ ID NO: 18 - DNA, 39 bp, Artificial sequence
  Primer
taatacgactcactataggaatttctactgttgtagatg

SEQ ID NO: 19 - DNA, 59 bp, Artificial sequence
  Primer
ttaaataaataaatccgatacatctacaacagtagaaattcctatagtgagtcgtatta

SEQ ID NO: 20 - DNA, 59 bp, Artificial sequence
  Primer
taaataaataaatccgataacatctacaacagtagaaattcctatagtgagtcgtatta

SEQ ID NO: 21 - DNA, 59 bp, Artificial sequence
  Primer
taataaataaatccgataaacatctacaacagtagaaattcctatagtgagtcgtatta

SEQ ID NO: 22 - DNA, 59 bp, Artificial sequence
  Primer
aataaataaatccgataaatcatctacaacagtagaaattcctatagtgagtcgtatta

SEQ ID NO: 23 - DNA, 59 bp, Artificial sequence
  Primer
ataaataaatccgataaatacatctacaacagtagaaattcctatagtgagtcgtatta

SEQ ID NO: 24 - DNA, 59 bp, Artificial sequence
  Primer
taaataaatccgataaataacatctacaacagtagaaattcctatagtgagtcgtatta

SEQ ID NO: 25 - DNA, 59 bp, Artificial sequence
  Primer
aaataaatccgataaataatcatctacaacagtagaaattcctatagtgagtcgtatta

SEQ ID NO: 26 - DNA, 59 bp, Artificial sequence
  Primer
aataaatccgataaataatacatctacaacagtagaaattcctatagtgagtcgtatta

SEQ ID NO: 27 - DNA, 59 bp, Artificial sequence
  Primer
ataaatccgataataataatcatctacaacagtagaaattcctatagtgagtcgtatta

SEQ ID NO: 28 - DNA, 59 bp, Artificial sequence
  Primer
taaatccgataaatataatacatctacaacagtagaaattcctatagtgagtcgtatta

SEQ ID NO: 29 - DNA, 59 bp, Artificial sequence
  Primer
aaatccgataaatataataacatctacaacagtagaaattcctatagtgagtcgtatta

SEQ ID NO: 30 - DNA, 59 bp, Artificial sequence
  Primer
aatccgataaatataataatcatctacaacagtagaaattcctatagtgagtcgtatta

SEQ ID NO: 31 - DNA, 59 bp, Artificial sequence
  Primer
atccgataaatataataatacatctacaacagtagaaattcctatagtgagtcgtatta

SEQ ID NO: 32 - DNA, 59 bp, Artificial sequence
  Primer
tccgatataataataataatcatctacaacagtagaaattcctatagtgagtcgtatta

SEQ ID NO: 33 - DNA, 59 bp, Artificial sequence
  Primer
gatataataataataataatcatctacaacagtagaaattcctatagtgagtcgtatta

SEQ ID NO: 34 - DNA, 59 bp, Artificial sequence
  Primer
ttaaataaataaatcctatacatctacaacagtagaaattcctatagtgagtcgtatta

SEQ ID NO: 35 - DNA, 59 bp, Artificial sequence
  Primer
taatacgactcactataggttatttcgtggatttatttagttttagagctagaaatagc

SEQ ID NO: 36 - DNA, 59 bp, Artificial sequence; identical to SEQ ID NO: 35
  Primer

SEQ ID NO: 37 - DNA, 59 bp, Artificial sequence; identical to SEQ ID NO: 35
  Primer

SEQ ID NO: 38 - DNA, 59 bp, Artificial sequence; identical to SEQ ID NO: 35
  Primer

SEQ ID NO: 39 - DNA, 74 bp, Artificial sequence
  substrate
  modified_base (37)..(37): n is m5c
  misc_feature (37)..(37): n is a, c, g, or t
ggtagttaggatgaatggaaggttggtatagcctatnggatttatttatttaatggatgacctctggatccatg

SEQ ID NO: 40 - DNA, 74 bp, Artificial sequence
  substrate
  modified_base (38)..(38): n is m5c
  misc_feature (38)..(38): n is a, c, g, or t
ggtagttaggatgaatggaaggttggtatagccttatnggatttatttatttatggatgacctctggatccatg

SEQ ID NO: 41 - DNA, 74 bp, Artificial sequence
  substrate
  modified_base (39)..(39): n is m5c
  misc_feature (39)..(39): n is a, c, g, or t
ggtagttaggatgaatggaaggttggtatagcctttatnggatttatttattatggatgacctctggatccatg

SEQ ID NO: 42 - DNA, 74 bp, Artificial sequence
  substrate
  modified_base (40)..(40): n is m5c
  misc_feature (40)..(40): n is a, c, g, or t
ggtagttaggatgaatggaaggttggtatagccatttatnggatttatttatttggatgacctctggatccatg

SEQ ID NO: 43 - DNA, 74 bp, Artificial sequence
  substrate
  modified_base (41)..(41): n is m5c
  misc_feature (41)..(41): n is a, c, g, or t
ggtagttaggatgaatggaaggttggtatagcctatttatnggatttatttattggatgacctctggatccatg

SEQ ID NO: 44 - DNA, 74 bp, Artificial sequence
  substrate
  modified_base (42)..(42): n is m5c
  misc_feature (42)..(42): n is a, c, g, or t
ggtagttaggatgaatggaaggttggtatagccttatttatnggatttatttatggatgacctctggatccatg

SEQ ID NO: 45 - DNA, 74 bp, Artificial sequence
  substrate
  modified_base (43)..(43): n is m5c
  misc_feature (43)..(43): n is a, c, g, or t
ggtagttaggatgaatggaaggttggtatagccattatttatnggatttattttggatgacctctggatccatg

SEQ ID NO: 46 - DNA, 74 bp, Artificial sequence
  substrate
  modified_base (44)..(44): n is m5c
  misc_feature (44)..(44): n is a, c, g, or t
ggtagttaggatgaatggaaggttggtatagcctattatttatnggatttatttggatgacctctggatccatg

SEQ ID NO: 47 - DNA, 74 bp, Artificial sequence
  substrate
  modified_base (45)..(45): n is m5c
  misc_feature (45)..(45): n is a, c, g, or t
ggtagttaggatgaatggaaggttggtatagccattattattatnggatttattggatgacctctggatccatg

SEQ ID NO: 48 - DNA, 74 bp, Artificial sequence
  substrate
  modified_base (46)..(46): n is m5c
  misc_feature (46)..(46): n is a, c, g, or t
ggtagttaggatgaatggaaggttggtatagcctattatatttatnggatttatggatgacctctggatccatg

SEQ ID NO: 49 - DNA, 74 bp, Artificial sequence
  substrate
  modified_base (47)..(47): n is m5c
  misc_feature (47)..(47): n is a, c, g, or t
ggtagttaggatgaatggaaggttggtatagccttattatatttatnggattttggatgacctctggatccatg

SEQ ID NO: 50 - DNA, 74 bp, Artificial sequence
  substrate
  modified_base (48)..(48): n is m5c
  misc_feature (48)..(48): n is a, c, g, or t
ggtagttaggatgaatggaaggttggtatagccattattatatttatnggatttggatgacctctggatccatg

SEQ ID NO: 51 - DNA, 74 bp, Artificial sequence
  substrate
  modified_base (49)..(49): n is m5c
  misc_feature (49)..(49): n is a, c, g, or t
ggtagttaggatgaatggaaggttggtatagcctattattatatttatnggattggatgacctctggatccatg

SEQ ID NO: 52 - DNA, 74 bp, Artificial sequence
  substrate
  modified_base (50)..(50): n is m5c
  misc_feature (50)..(50): n is a, c, g, or t
ggtagttaggatgaatggaaggttggtatagccattattattattatatnggatggatgacctctggatccatg

SEQ ID NO: 53 - DNA, 74 bp, Artificial sequence
  substrate
  modified_base (53)..(53): n is m5c
  misc_feature (53)..(53): n is a, c, g, or t
ggtagttaggatgaatggaaggttggtatagccattattattattattatatntggatgacctctggatccatg

SEQ ID NO: 54 - DNA, 74 bp, Artificial sequence
  In vitro reaction substrate
ggtagttaggatgaatggaaggttggtatagcctataggatttatttatttaatggatgacctctggatccatg

SEQ ID NO: 55 - DNA, 74 bp, Artificial sequence
  In vitro reaction substrate
catggatccagaggtcatccattaaataaataaatccgataggctataccaaccttccattcatcctaactacc

SEQ ID NO: 56 - DNA, 74 bp, Artificial sequence
  In vitro reaction substrate
catggatccagaggtcatccataaataaataaatccgataaggctataccaaccttccattcatcctaactacc

SEQ ID NO: 57 - DNA, 74 bp, Artificial sequence
  In vitro reaction substrate
catggatccagaggtcatccataataaataaatccgataaaggctataccaaccttccattcatcctaactacc

SEQ ID NO: 58 - DNA, 74 bp, Artificial sequence
  In vitro reaction substrate
catggatccagaggtcatccaaataaataaatccgataaatggctataccaaccttccattcatcctaactacc

SEQ ID NO: 59 - DNA, 74 bp, Artificial sequence
  In vitro reaction substrate
catggatccagaggtcatccaataaataaatccgataaataggctataccaaccttccattcatcctaactacc

SEQ ID NO: 60 - DNA, 74 bp, Artificial sequence
  In vitro reaction substrate
catggatccagaggtcatccataaataaatccgataaataaggctataccaaccttccattcatcctaactacc

SEQ ID NO: 61 - DNA, 74 bp, Artificial sequence
  In vitro reaction substrate
catggatccagaggtcatccaaaataaatccgataaataatggctataccaaccttccattcatcctaactacc

SEQ ID NO: 62 - DNA, 74 bp, Artificial sequence
  In vitro reaction substrate
catggatccagaggtcatccaaataaatccgataaataataggctataccaaccttccattcatcctaactacc

SEQ ID NO: 63 - DNA, 74 bp, Artificial sequence
  In vitro reaction substrate
catggatccagaggtcatccaataaatccgataataataatggctataccaaccttccattcatcctaactacc

SEQ ID NO: 64 - DNA, 74 bp, Artificial sequence
  In vitro reaction substrate
catggatccagaggtcatccataaatccgataaatataataggctataccaaccttccattcatcctaactacc

SEQ ID NO: 65 - DNA, 74 bp, Artificial sequence
  In vitro reaction substrate
catggatccagaggtcatccaaaatccgataaatataataaggctataccaaccttccattcatcctaactacc

SEQ ID NO: 66 - DNA, 74 bp, Artificial sequence
  In vitro reaction substrate
catggatccagaggtcatccaaatccgataaatataataatggctataccaaccttccattcatcctaactacc

SEQ ID NO: 67 - DNA, 74 bp, Artificial sequence
  In vitro reaction substrate
catggatccagaggtcatccaatccgataaatataataataggctataccaaccttccattcatcctaactacc

SEQ ID NO: 68 - DNA, 74 bp, Artificial sequence
  In vitro reaction substrate
catggatccagaggtcatccatccgatataataataataatggctataccaaccttccattcatcctaactacc

SEQ ID NO: 69 - DNA, 74 bp, Artificial sequence
  In vitro reaction substrate
catggatccagaggtcatccagatataataataataataatggctataccaaccttccattcatcctaactacc

SEQ ID NO: 70 - DNA, 74 bp, Artificial sequence
  In vitro reaction substrate
catggatccagaggtcatccattaaataaataaatcctataggctataccaaccttccattcatcctaactacc

SEQ ID NO: 71 - DNA, 59 bp, Artificial sequence
  substrate
  modified_base (23)..(23): n is m5c
  misc_feature (23)..(23): n is a, c, g, or t
ggtacccggggatcctttatatnggatttatttatttaagttaaaaagcttggcgtaat

SEQ ID NO: 72 - DNA, 59 bp, Artificial sequence
  substrate
  modified_base (24)..(24): n is m5c
  misc_feature (24)..(24): n is a, c, g, or t
ggtacccggggatcctttattatnggatttatttatttagttaaaaagcttggcgtaat

SEQ ID NO: 73 - DNA, 59 bp, Artificial sequence
  substrate
  modified_base (25)..(25): n is m5c
  misc_feature (25)..(25): n is a, c, g, or t
ggtacccggggatcctttatttatnggatttatttattagttaaaaagcttggcgtaat

SEQ ID NO: 74 - DNA, 59 bp, Artificial sequence
  substrate
  modified_base (26)..(26): n is m5c
  misc_feature (26)..(26): n is a, c, g, or t
ggtacccggggatcctttaatttatnggatttatttattgttaaaaagcttggcgtaat

SEQ ID NO: 75 - DNA, 59 bp, Artificial sequence
  substrate
  modified_base (27)..(27): n is m5c
  misc_feature (27)..(27): n is a, c, g, or t
ggtacccggggatcctttatatttatnggatttatttatgttaaaaagcttggcgtaat

SEQ ID NO: 76 - DNA, 59 bp, Artificial sequence
  substrate
  modified_base (28)..(28): n is m5c
  misc_feature (28)..(28): n is a, c, g, or t
ggtacccggggatcctttattatttatnggatttatttagttaaaaagcttggcgtaat

SEQ ID NO: 77 - DNA, 59 bp, Artificial sequence
  substrate
  modified_base (29)..(29): n is m5c
  misc_feature (29)..(29): n is a, c, g, or t
ggtacccggggatcctttaattatttatnggatttatttgttaaaaagcttggcgtaat

SEQ ID NO: 78 - DNA, 59 bp, Artificial sequence
  substrate
  modified_base (30)..(30): n is m5c
  misc_feature (30)..(30): n is a, c, g, or t
ggtacccggggatcctttatattatttatnggatttattgttaaaaagcttggcgtaat

SEQ ID NO: 79 - DNA, 59 bp, Artificial sequence
  substrate
  modified_base (31)..(31): n is m5c
  misc_feature (31)..(31): n is a, c, g, or t
ggtacccggggatcctttaattattattatnggatttatgttaaaaagcttggcgtaat

SEQ ID NO: 80 - DNA, 59 bp, Artificial sequence
  substrate
  modified_base (32)..(32): n is m5c
  misc_feature (32)..(32): n is a, c, g, or t
ggtacccggggatcctttatattatatttatnggatttagttaaaaagcttggcgtaat

SEQ ID NO: 81 - DNA, 59 bp, Artificial sequence
  substrate
  modified_base (33)..(33): n is m5c
  misc_feature (33)..(33): n is a, c, g, or t
ggtacccggggatcctttattattatatttatnggatttgttaaaaagcttggcgtaat

SEQ ID NO: 82 - DNA, 59 bp, Artificial sequence
  substrate
  modified_base (34)..(34): n is m5c
  misc_feature (34)..(34): n is a, c, g, or t
ggtacccggggatcctttaattattatatttatnggattgttaaaaagcttggcgtaat

SEQ ID NO: 83 - DNA, 59 bp, Artificial sequence
  substrate
  modified_base (35)..(35): n is m5c
  misc_feature (35)..(35): n is a, c, g, or t
ggtacccggggatcctttatattattatatttatnggatgttaaaaagcttggcgtaat

SEQ ID NO: 84 - DNA, 59 bp, Artificial sequence
  substrate
  modified_base (36)..(36): n is m5c
  misc_feature (36)..(36): n is a, c, g, or t
ggtacccggggatcctttaattattattattatatnggagttaaaaagcttggcgtaat

SEQ ID NO: 85 - DNA, 59 bp, Artificial sequence
  substrate
  modified_base (39)..(39): n is m5c
  misc_feature (39)..(39): n is a, c, g, or t
ggtacccggggatcctttaattattattattattatatngttaaaaagcttggcgtaat

SEQ ID NO: 86 - DNA, 59 bp, Artificial sequence
  In vitro reaction substrate
attacgccaagctttttaacttaaataaataaatccgatataaaggatccccgggtacc

SEQ ID NO: 87 - DNA, 59 bp, Artificial sequence
  In vitro reaction substrate
attacgccaagctttttaactaaataaataaatccgataataaaggatccccgggtacc

SEQ ID NO: 88 - DNA, 59 bp, Artificial sequence
  In vitro reaction substrate
attacgccaagctttttaactaataaataaatccgataaataaaggatccccgggtacc

SEQ ID NO: 89 - DNA, 59 bp, Artificial sequence
  In vitro reaction substrate
attacgccaagctttttaacaataaataaatccgataaattaaaggatccccgggtacc

SEQ ID NO: 90 - DNA, 59 bp, Artificial sequence
  In vitro reaction substrate
attacgccaagctttttaacataaataaatccgataaatataaaggatccccgggtacc

SEQ ID NO: 91 - DNA, 59 bp, Artificial sequence
  In vitro reaction substrate
attacgccaagctttttaactaaataaatccgataaataataaaggatccccgggtacc

SEQ ID NO: 92 - DNA, 59 bp, Artificial sequence
  In vitro reaction substrate
attacgccaagctttttaacaaataaatccgataaataattaaaggatccccgggtacc

SEQ ID NO: 93 - DNA, 59 bp, Artificial sequence
  In vitro reaction substrate
attacgccaagctttttaacaataaatccgataaataatataaaggatccccgggtacc

SEQ ID NO: 94 - DNA, 59 bp, Artificial sequence
  In vitro reaction substrate
attacgccaagctttttaacataaatccgataataataattaaaggatccccgggtacc

SEQ ID NO: 95 - DNA, 59 bp, Artificial sequence
  In vitro reaction substrate
attacgccaagctttttaactaaatccgataaatataatataaaggatccccgggtacc

SEQ ID NO: 96 - DNA, 59 bp, Artificial sequence
  In vitro reaction substrate
attacgccaagctttttaacaaatccgataaatataataataaaggatccccgggtacc

SEQ ID NO: 97 - DNA, 59 bp, Artificial sequence
  In vitro reaction substrate
attacgccaagctttttaacaatccgataaatataataattaaaggatccccgggtacc

SEQ ID NO: 98 - DNA, 59 bp, Artificial sequence
  In vitro reaction substrate
attacgccaagctttttaacatccgataaatataataatataaaggatccccgggtacc

SEQ ID NO: 99 - DNA, 59 bp, Artificial sequence
  In vitro reaction substrate
attacgccaagctttttaactccgatataataataataattaaaggatccccgggtacc

SEQ ID NO: 100 - DNA, 59 bp, Artificial sequence
  In vitro reaction substrate
attacgccaagctttttaacgatataataataataataattaaaggatccccgggtacc

SEQ ID NO: 101 - DNA, 59 bp, Artificial sequence
  substrate
  modified_base (27)..(27): n is m5c
  misc_feature (27)..(27): n is a, c, g, or t
acgtaaacggccacaagttcttatttngtggatttatttatggcatcttcttcaaggac

SEQ ID NO: 102 - DNA, 59 bp, Artificial sequence
  substrate
  modified_base (27)..(27): n is m5c
  misc_feature (27)..(27): n is a, c, g, or t
acgtaaacggccacaagttcttattangtggatttatttatggcatcttcttcaaggac

SEQ ID NO: 103 - DNA, 59 bp, Artificial sequence
  substrate
  modified_base (27)..(27): n is m5c
  misc_feature (27)..(27): n is a, c, g, or t
acgtaaacggccacaagttcttattcngtggatttatttatggcatcttcttcaaggac

SEQ ID NO: 104 - DNA, 59 bp, Artificial sequence
  substrate
  modified_base (27)..(27): n is m5c
  misc_feature (27)..(27): n is a, c, g, or t
acgtaaacggccacaagttcttattgngtggatttatttatggcatcttcttcaaggac

SEQ ID NO: 105 - DNA, 59 bp, Artificial sequence
  In vitro reaction substrate
gtccttgaagaagatgccataaataaatccacgaaataagaacttgtggccgtttacgt

SEQ ID NO: 106 - DNA, 59 bp, Artificial sequence
  In vitro reaction substrate
gtccttgaagaagatgccataaataaatccacgtaataagaacttgtggccgtttacgt

SEQ ID NO: 107 - DNA, 59 bp, Artificial sequence
  In vitro reaction substrate
gtccttgaagaagatgccataaataaatccacggaataagaacttgtggccgtttacgt

SEQ ID NO: 108 - DNA, 59 bp, Artificial sequence
  In vitro reaction substrate
gtccttgaagaagatgccataaataaatccacgcaataagaacttgtggccgtttacgt

SEQ ID NO: 109 - DNA, 23 bp, Artificial sequence
  Primer
cgtaaacggccacaagttcttat

SEQ ID NO: 110 - DNA, 23 bp, Artificial sequence
  Primer
gtccttgaagaagatgccataaa

SEQ ID NO: 111 - DNA, 17 bp, Artificial sequence
  Primer
cggccacaagttcttat

SEQ ID NO: 112 - DNA, 26 bp, Artificial sequence
  Primer
ggatttgygttttttygaagatttgg

SEQ ID NO: 113 - DNA, 30 bp, Artificial sequence
  Primer
aaatacraatactcttcraatttcaaaaac

SEQ ID NO: 114 - DNA, 20 bp, Artificial sequence
  Primer
gttttttagaagatttggat

SEQ ID NO: 115 - DNA, 30 bp, Artificial sequence
  Primer
gttttgaatgaatgtgtgtatatatgtatg

SEQ ID NO: 116 - DNA, 30 bp, Artificial sequence
  Primer
ctaacaaaaaccaaactaattcttatctac

SEQ ID NO: 117 - DNA, 25 bp, Artificial sequence
  Primer
atgaatgtgtgtatatatgtatgag

SEQ ID NO: 118 - DNA, 21 bp, Artificial sequence
  Primer
taagggttttyggaayggggt

SEQ ID NO: 119 - DNA, 21 bp, Artificial sequence
  Primer
ccaaacaaaacatccctcaac

SEQ ID NO: 120 - DNA, 16 bp, Artificial sequence
  Primer
gggttgtgtgagtggg

SEQ ID NO: 121 - DNA, 25 bp, Artificial sequence
  Primer
caccgggacccgcgcctgatgcacg

SEQ ID NO: 122 - DNA, 25 bp, Artificial sequence
  Primer
aaaccgtgcatcaggcgcgggtccc

SEQ ID NO: 123 - DNA, 25 bp, Artificial sequence
  Primer
caccggagctggcggcagtcggggt

SEQ ID NO: 124 - DNA, 25 bp, Artificial sequence
  Primer
aaacaccccgactgccgccagctcc

SEQ ID NO: 125 - DNA, 25 bp, Artificial sequence
  Primer
caccgttccgagaagtctattgagc

SEQ ID NO: 126 - DNA, 25 bp, Artificial sequence
  Primer
aaacgctcaatagacttctcggaac

SEQ ID NO: 127 - DNA, 25 bp, Artificial sequence
  Primer
caccgtggggccgtcgggccgggct

SEQ ID NO: 128 - DNA, 25 bp, Artificial sequence
  Primer
aaacagcccggcccgacggccccac

SEQ ID NO: 129 - DNA, 25 bp, Artificial sequence
  Primer
caccgtcagccggggctagaaaagg

SEQ ID NO: 130 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 131 - protein, 182 aa, Homo sapiens [sequence omitted]

SEQ ID NO: 132 - protein, 1376 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPPKKKRKV

SEQ ID NO: 133 - protein, 1307 aa, Acidaminococcus fermentans
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 134-200 - skipped (67 entries)

SEQ ID NO: 201 - protein, 1608 aa, Artificial sequence
  Recombinant protein
HHHHHHSSGLVPRGSHMPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNGGSGGSGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPPKKKRKV

SEQ ID NO: 202 - protein, 1619 aa, Artificial sequence
  Recombinant protein
HHHHHHSSGLVPRGSHMPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNGGSGGSGGSGGSGGSGGSGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPPKKKRKV

SEQ ID NO: 203 - protein, 1640 aa, Artificial sequence
  Recombinant protein
HHHHHHSSGLVPRGSHMPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNGGSGGSGGSGGSGGSGGSGGSGGSGGSGGSGGSGGSGGSGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPPKKKRKV

SEQ ID NO: 204 - protein, 1591 aa, Artificial sequence
  Recombinant protein
HHHHHHSSGLVPRGSHMPKKKRKVMALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLRIFASRLYYHWCKPQQDGLRLLCGSQVPVEVMGFPEFADCWENFVDHEKPLSFNPYKMLEELDKNSRAIKRRLDRIKSGGSGGSGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPPKKKRKV

SEQ ID NO: 205 - protein, 1603 aa, Artificial sequence
  Recombinant protein
HHHHHHSSGLVPRGSHMPKKKRKVMALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLRIFASRLYYHWCKPQQDGLRLLCGSQVPVEVMGFPEFADCWENFVDHEKPLSFNPYKMLEELDKNSRAIKRRLDRIKSGGSGGSGGSGGSGGSGGSGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPPKKKRKV

SEQ ID NO: 206 - protein, 1624 aa, Artificial sequence
  Recombinant protein
HHHHHHSSGLVPRGSHMPKKKRKVMALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLRIFASRLYYHWCKPQQDGLRLLCGSQVPVEVMGFPEFADCWENFVDHEKPLSFNPYKMLEELDKNSRAIKRRLDRIKSGGSGGSGGSGGSGGSGGSGGSGGSGGSGGSGGSGGSGGSGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPPKKKRKV

SEQ ID NO: 207 - protein, 1535 aa, Artificial sequence
  Recombinant protein
HHHHHHSSGLVPRGSHMPKKKRKVMALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLRIFASRLYYHWCKPQQDGLRLLCGSQVPVEVMGFPEFADCWENFVDHEKPLSFNPYKMLEELDKNSRAIKRRLDRIKSGGSGGSGGSGGSGGSGGSGGSKLTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 208-300 - skipped (93 entries)

SEQ ID NO: 301 - DNA, 4821 bp, Artificial sequence
  DNA sequence encoding recombinant protein
atgggcagcagccatcatcatcatcatcacagcagcggcctggtgccgcgcggcagccatatgccaaagaagaagcggaaggtcgaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcgcagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacggaggaagtggaggaagtggaggaagtaagcttgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactaactcgag

SEQ ID NO: 302 - DNA, 4857 bp, Artificial sequence
  DNA sequence encoding recombinant protein
atgggcagcagccatcatcatcatcatcacagcagcggcctggtgccgcgcggcagccatatgccaaagaagaagcggaaggtcgaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcgcagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacggaggaagtggaggaagtggaggaagtggaggaagtggaggaagtggaggaagtggaggaagtaagcttgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactaactcgag

SEQ ID NO: 303 - DNA, 4920 bp, Artificial sequence
  DNA sequence encoding recombinant protein
atgggcagcagccatcatcatcatcatcacagcagcggcctggtgccgcgcggcagccatatgccaaagaagaagcggaaggtcgaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcgcagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacggaggaagtggaggaagtggaggaagtggaggaagtggaggaagtggaggaagtggaggaagtggaggaagtggaggaagtggaggaagtggaggaagtggaggaagtggaggaagtggaggaagtaagcttgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactaactcgag

SEQ ID NO: 304 - DNA, 4769 bp, Artificial sequence
  DNA sequence encoding recombinant protein
atgggcagcagccatcatcatcatcatcacagcagcggcctggtgccgcgcggcagccatatgccaaagaagaagcggaaggtcgctcttcttactgctgaaacttttcgtctccaatttaataataaacgccgtctgcgtcgcccgtattacccgcgcaaggcgctgctgtgttaccaactgaccccacaaaacggttccaccccgactcgcggttactttgagaataagaaaaaatgtcacgctgagatctgtttcattaacgaaatcaaatctatgggcctggatgaaactcagtgctaccaggtcacctgctacctgacctggagcccgtgtagctcttgcgcgtgggaactggttgacttcatcaaagcgcacgaccatctgaacctgcgtatcttcgcttcccgcctgtactatcactggtgcaagccgcaacaggatggcctgcgcctgctgtgtggttctcaggttccggttgaagttatgggtttcccggagtttgcggactgctgggaaaactttgttgaccatgagaagccactgtcctttaacccgtataaaatgctggaagagctggacaaaaactctcgtgctatcaagcgccgtctggatcgtatcaagtctggaggaagtggaggaagtggaggaagtagcttgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactaactcgag

SEQ ID NO: 305 - DNA, 4806 bp, Artificial sequence
  DNA sequence encoding recombinant protein
atgggcagcagccatcatcatcatcatcacagcagcggcctggtgccgcgcggcagccatatgccaaagaagaagcggaaggtcgctcttcttactgctgaaacttttcgtctccaatttaataataaacgccgtctgcgtcgcccgtattacccgcgcaaggcgctgctgtgttaccaactgaccccacaaaacggttccaccccgactcgcggttactttgagaataagaaaaaatgtcacgctgagatctgtttcattaacgaaatcaaatctatgggcctggatgaaactcagtgctaccaggtcacctgctacctgacctggagcccgtgtagctcttgcgcgtgggaactggttgacttcatcaaagcgcacgaccatctgaacctgcgtatcttcgcttcccgcctgtactatcactggtgcaagccgcaacaggatggcctgcgcctgctgtgtggttctcaggttccggttgaagttatgggtttcccggagtttgcggactgctgggaaaactttgttgaccatgagaagccactgtcctttaacccgtataaaatgctggaagagctggacaaaaactctcgtgctatcaagcgccgtctggatcgtatcaagtctggaggaagtggaggaagtggaggaagtggaggaagtggaggaagtggaggaagtggaggaagtaagcttgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactaactcgag

SEQ ID NO: 306 - DNA, 4869 bp, Artificial sequence
  DNA sequence encoding recombinant protein
atgggcagcagccatcatcatcatcatcacagcagcggcctggtgccgcgcggcagccatatgccaaagaagaagcggaaggtcgctcttcttactgctgaaacttttcgtctccaatttaataataaacgccgtctgcgtcgcccgtattacccgcgcaaggcgctgctgtgttaccaactgaccccacaaaacggttccaccccgactcgcggttactttgagaataagaaaaaatgtcacgctgagatctgtttcattaacgaaatcaaatctatgggcctggatgaaactcagtgctaccaggtcacctgctacctgacctggagcccgtgtagctcttgcgcgtgggaactggttgacttcatcaaagcgcacgaccatctgaacctgcgtatcttcgcttcccgcctgtactatcactggtgcaagccgcaacaggatggcctgcgcctgctgtgtggttctcaggttccggttgaagttatgggtttcccggagtttgcggactgctgggaaaactttgttgaccatgagaagccactgtcctttaacccgtataaaatgctggaagagctggacaaaaactctcgtgctatcaagcgccgtctggatcgtatcaagtctggaggaagtggaggaagtggaggaagtggaggaagtggaggaagtggaggaagtggaggaagtggaggaagtggaggaagtggaggaagtggaggaagtggaggaagtggaggaagtggaggaagtaagcttgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactaactcgag

SEQ ID NO: 307 - DNA, 4750 bp, Artificial sequence
  DNA sequence encoding recombinant protein
atgggcagcagccatcatcatcatcatcacagcagcggcctggtgccgcgcggcagccatatgccaaagaagaagcggaaggtcgctcttcttactgctgaaacttttcgtctccaatttaataataaacgccgtctgcgtcgcccgtattacccgcgcaaggcgctgctgtgttaccaactgaccccacaaaacggttccaccccgactcgcggttactttgagaataagaaaaaatgtcacgctgagatctgtttcattaacgaaatcaaatctatgggcctggatgaaactcagtgctaccaggtcacctgctacctgacctggagcccgtgtagctcttgcgcgtgggaactggttgacttcatcaaagcgcacgaccatctgaacctgcgtatcttcgcttcccgcctgtactatcactggtgcaagccgcaacaggatggcctgcgcctgctgtgtggttctcaggttccggttgaagttatgggtttcccggagtttgcggactgctgggaaaactttgttgaccatgagaagccactgtcctttaacccgtataaaatgctggaagagctggacaaaaactctcgtgctatcaagcgccgtctggatcgtatcaagtctggaggaagtggaggaagtggaggaagtggaggaagtggaggaagtggaggaagtggaggaagtatgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacaaaaggccggcggccacgaaaaaggccggccaggcaaaaaagaaaaagggatcctacccatacgatgttccagattacgcttatccctacgacgtgcctgattatgcatacccatatgatgtccccgactatgcctaag

SEQ ID NO: 308 - DNA, 23 bp, Artificial sequence
  substrate
  modified_base (7)..(7): n is methylated-C
  misc_feature (7)..(7): n is a, c, g, or t
ttattgngtggatttatttatgg

SEQ ID NO: 309 - DNA, 23 bp, Artificial sequence
  substrate
  misc_feature (7)..(7): n is methylated-C
ttattcngtggatttatttatgg

SEQ ID NO: 310 - DNA, 23 bp, Artificial sequence
  substrate
  modified_base (7)..(7): n is methylated-C
  misc_feature (7)..(7): n is a, c, g, or t
ttattangtggatttatttatgg

SEQ ID NO: 311 - DNA, 23 bp, Artificial sequence
  substrate
  modified_base (7)..(7): n is methylated-C
  misc_feature (7)..(7): n is a, c, g, or t
ttatttngtggatttatttatgg

SEQ ID NO: 312 - DNA, 20 bp, artificial sequence
  substrate
  modified_base (6)..(6): n is 5mC
  misc_feature (6)..(6): n is a, c, g, or t
  modified_base (8)..(8): n is 5mC
  misc_feature (8)..(8): n is a, c, g, or t
  modified_base (19)..(19): n is 5mC
  misc_feature (19)..(19): n is a, c, g, or t
ggaccngngcctgatgcang

SEQ ID NO: 313 - DNA, 20 bp, artificial sequence
  substrate
  modified_base (8)..(8): n is 5mC
  misc_feature (8)..(8): n is a, c, g, or t
  modified_base (15)..(15): n is 5mC
  misc_feature (15)..(15): n is a, c, g, or t
gagctggnggcagtnggggt

SEQ ID NO: 314 - DNA, 20 bp, artificial sequence
  substrate
  modified_base (7)..(7): n is 5mC
  misc_feature (7)..(7): n is a, c, g, or t
  modified_base (10)..(10): n is 5mC
  misc_feature (10)..(10): n is a, c, g, or t
tggggcngtngggccgggct

SEQ ID NO: 315 - DNA, 20 bp, artificial sequence
  substrate
  modified_base (7)..(7): n is 5mC
  misc_feature (7)..(7): n is a, c, g, or t
  modified_base (10)..(10): n is 5mC
  misc_feature (10)..(10): n is a, c, g, or t
tggggcngtngggccgggct

(19) United States
(12) Patent Application Publication
(10) Pub. No.: US 2023/0151341 A1
(43) Pub. Date: May 18, 2023

(54) METHOD FOR SPECIFICALLY EDITING GENOMIC DNA AND APPLICATION THEREOF

(71) Applicant: Qihan CHEN, Nanjing (CN)

(72) Inventor: Qihan CHEN, Nanjing (CN)

(21) Appl. No.: 16/317,524

(22) PCT Filed: Jun. 14, 2017
(86) PCT No.: PCT/CN2017/088281

§ 371 Date: Jan. 11, 2019

(30) Foreign Application Priority Data
Jul. 13, 2016 (CN) 201610550293.X

(51) Int. Cl. C12N 9/22; C12N 15/11; C12N 9/78; C12N 15/90; A61K 48/00

(52) U.S. Cl. CPC C12N 9/22; C12N 15/11; C12N 9/78; C12Y 305/04; C12N 15/90; A61K 48/0091; C12N 2310/20; C12N 2800/80

(57) ABSTRACT

A method for modulating a methylation/demethylation state of a nucleic acid, more specifically, a method for site-removing one or more methylated bases from a genome guided by a sgRNA sequence in a cell.

### FIELD OF THE INVENTION

The present invention relates to the field of bioengineering technology, and in particular relates to a method for specifically modulating the methylation/demethylation status of genomic DNA and use thereof.

### BACKGROUND OF THE INVENTION

DNA methylation is one of the important modifications in epigenetic modulation and is called the “fifth base” in mammalian DNA except for the four bases of ATCG. As a covalent modification, DNA methylation plays an important role in normal differentiation and disease development and can be stably inherited in cell differentiation of higher eukaryotic organs, and it is found in zebrafish that DNA methylation can be passed on to the next generation through sperm. Under the influence of cell differentiation, disease and environment, the methylation status of DNA will change greatly.

Studies have shown that DNA methylation is closely related to the occurrence and development of tumors. Changes in DNA methylation status include hypermethylation and hypomethylation. In general, DNA hypermethylation in the promoter region of the gene has the effect of silencing gene expression, while hypomethylation activates gene expression. DNA analysis of different tumor cells showed that the probability of genetic mutations in cancerous cells was much lower than expected. In the transcriptome range, gene expression inhibition by promoter hypermethylation in colorectal cancer was detected, and it was found that up to 5% of known genes have abnormal promoter hypermethylation in tumor cells. Therefore, it can be speculated that DNA methylation changes may play a greater role in cell malignant transformation than genetic mutations.

Target-specific nucleic acid editing techniques, especially the specific editing of genomic DNA, have always been an important technical basis for gene therapy. With the deepening of epigenetics research, more and more studies have shown that the methylation of the genome is directly involved in transcriptional modulation and other modulation of the genome, while the promotor and enhancer regions of an active expression gene are usually hypomethylated. Therefore, a nucleotide editing technique capable of specific demethylation is very important for the transcriptional activation of silenced genes.

Currently, site-specific and region-specific demethylation processes have been reported. For example, genomic remodeling of germ cells is often accompanied by large-scale demethylation. In addition, 5mC can be oxidized by certain enzymes (such as Tet) to 5hmC, followed by NER or BER process to be finally demethylated. Xu Guoliang, et al., have reported and filed a patent application for demethylation by reagents such as Tet dioxygenase and thymidine DNA glycosylase in 2015, but this method has not been able to accurately edit a certain site, being an important bottleneck for use in gene therapy or experimental technology tools.

Certain members of the Apobec protein family have the ability to deaminate 5mC into T in single-stranded DNA. With such characteristics and the precise positioning ability of the CRISPR protein family, it has become possible to develop a system that can accurately edit methylation at a specific site in the genome.

### SUMMARY OF THE INVENTION

In order to solve the above problems, the present invention provides a method for editing a target nucleic acid molecule, comprising the steps of:
(1) obtaining a recombinant vector encoding a fusion protein (A) and a small guide RNA (sgRNA) (B), wherein the fusion protein (A) comprises an Apobec family protein domain at N-terminal and a Cas9 family or a Cpf1 family protein domain whose nuclease activity is inactivated at C-terminal, and the small guide RNA has a complementary region to a target editing region of the target nucleic acid molecule, wherein the target editing region of the target nucleic acid molecule includes at least one methylated cytosine nucleotide;
(2) contacting the recombinant vector encoding the fusion protein (A) and the small guide RNA (sgRNA) (B) obtained in the step (1) with the target nucleic acid molecule.

The recombinant vector in the above steps may be a recombinant vector in which two vectors respectively encode the fusion protein (A) and the small guide RNA (sgRNA) (B), or a recombinant vector in which a recombinant vector encodes both the fusion protein (A) and the small guide RNA (sgRNA) (B).

In a preferred embodiment, the Apobec family protein at N-terminal of the fusion protein is selected from the group consisting of human Apobec3A or Apobec3H, or a protein having deamination activity with 95% or more homology to human Apobec3A or Apobec3H. More preferably, the Apobec protein is Apobec3H or Apobec3A.

In another preferred embodiment, the Cas9 family protein whose nuclease activity is inactivated at C-terminal of the fusion protein is the one obtained by mutating aspartic acid at position 10 and histidine at position 840 in the wild-type Cas9 protein to alanine and alanine, or the Cpf1 protein whose nuclease activity is inactivated at C-terminal of the fusion protein is the one obtained by mutating aspartic acid to alanine at position 908 in the wide-type Cpf1 protein.

In order to provide better spatial structural flexibility for the two protein domains of the fusion protein, a linker consisting of 3-14 motifs can be added between the two domains of the fusion protein. The motif is selected from (GGS). The longer the linker is, the higher the spatial flexibility of the protein is and the larger the editable target area is.

To facilitate expression and purification of the fusion protein, a purification tag sequence can also be included. A commonly used purification tag is 6xHis.

In a more preferred embodiment, the fusion protein is selected from any of the sequences of SEQ ID NOs. 201-207.

The present invention also provides a gene sequence encoding the above fusion protein sequence, which is preferably selected from the group consisting of SEQ ID NOs. 301-307.

The present invention also provides a recombinant vector comprising any of the above gene sequences, which may be a prokaryotic expression vector or a eukaryotic expression vector, including but not limited to a plasmid vector, a viral vector, and the like, for the purpose of subsequent experiments.

Another aspect of the invention provides a small guide RNA molecule. In a preferred embodiment, the small guide RNA is 60 to 80 bp in length. In another preferred embodiment, the complementary region of the small guide RNA to the target nucleic acid molecule is 18 to 25 bp in length, preferably 20 bp.

A method for editing a target nucleic acid molecule in vitro, comprising the steps of: (1) obtaining a recombinant vector encoding a fusion protein (A) and a small guide RNA (sgRNA) (B), wherein the fusion protein (A) comprises an Apobec family protein domain at N-terminal and a Cas9 family or a Cpf1 family protein domain whose nuclease activity is inactivated at C-terminal, and the small guide RNA has a complementary region to a target editing region of the target nucleic acid molecule, wherein the target editing region of the target nucleic acid molecule includes at least one methylated cytosine nucleotide;
(2) contacting the fusion protein (A) and the small guide RNA (sgRNA) (B) with the target nucleic acid molecule;
(3) after a high temperature termination reaction, adding an effective amount of TDG, and carrying out a reaction at 42° C. for 6 to 8 hours; and
(4) adding an effective amount of EDTA, formamide and NaOH, and carrying out a reaction at 90 to 95° C. for 5 to 10 minutes.

The present invention also provides use of the method for editing a target nucleic acid molecule for specifically modulating genomic DNA methylation/demethylation status.

In the method for editing a target nucleic acid molecule according to the present invention, the target nucleic acid molecule contains at least one methylated cytosine nucleotide, the methylated cytidine nucleotide is associated with diseases such as cancer, genetic disorders, developmental errors and the like. The method for editing a target nucleic acid molecule can be used for the treatment of a disease associated with cytosine nucleotide methylation, including but not limited to diseases associated with abnormal cell differentiation.

### The Beneficial Effects of the Present Invention

In the present invention, the Apobec protein having deamination activity is guided to the methylated cytosine position of the target nucleic acid molecule to modify the methylated cytosine by the guidance of sgRNA and the specific binding function of the mutant Cas9 or Cpf1. Further, the methylated cytosine is removed by an in vivo DNA repair mechanism to achieve specific editing of the target nucleic acid molecule. The gene editing method of the present invention has high specificity and has no dependence on the upstream and downstream sequences of the target site, and thus has universal applicability. Moreover, the gene editing method of the present invention only edits the target, does not produce off-target effects, and does not introduce insertion or deletion mutations during editing, thus has low toxic side effects.

### BRIEF DESCRIPTION OF THE DRAWINGS

FIG. 1 shows a schematic diagram of extracellular editing of fusion protein.

FIG. 2 shows a schematic diagram of intracellular editing of fusion protein.

FIG. 3 shows tests for active intensities and ranges of several fusion proteins in vitro.

FIG. 4 shows effect of the base located adjacent to upstream of the editing target site on editing efficiency.

FIG. 5 shows editing results in two groups of HEK293 cell lines.

FIG. 6 shows editing results of the two fusion proteins in the same region of the PC3 cell line.

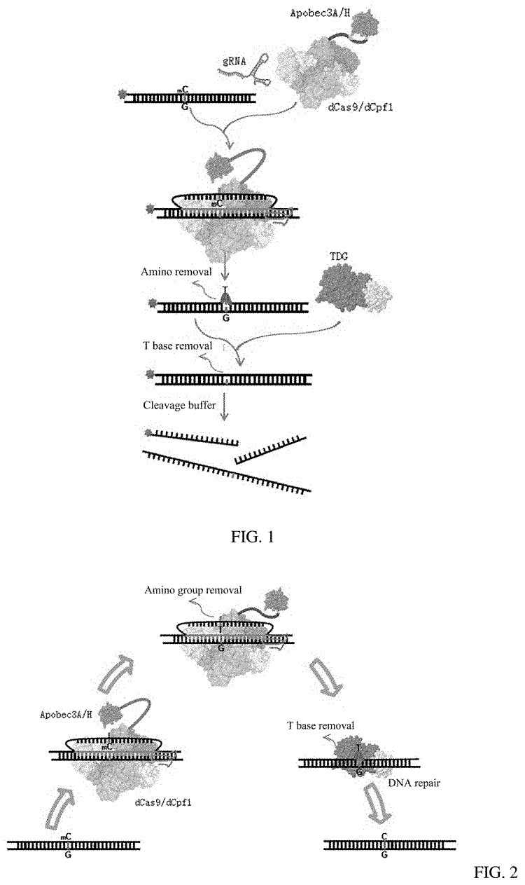

### DETAILED DESCRIPTION OF THE INVENTION

The Cas9 or Cpf1 protein is a double-stranded DNA nuclease that binds to a targeting sequence and cleaves double-stranded DNA under the action of a small guide RNA (sgRNA). The Cas9 protein whose nuclease activity is inactivated retains the activity of binding to the targeting sequence, but does not cleave the target site. In the present invention, the methylated cytosine in the targeted sequence region is deaminated by fusing the Cas9 or Cpf1 protein whose nuclease activity is inactivated with the Apobec protein having deamination activity and guiding the Apobec protein to the target sequence region of the target nucleic acid molecule by the mutated Cas9 protein or Cpf1 protein, so that the target Met-C becomes T under deamination and does not pair with G on the complementary chain to form a protrusion. The addition of an effective amount of TDG after termination of the reaction by high temperature (the main effect is to inactivate the fusion protein by high temperature, usually at a temperature of 90 to 95° C.) removes the mismatched T base, thereby forming a deletion at the editing target site of the substrate. The dsDNA then changes back to ssDNA and cleaves at the base deletion site by the combined action of an effective amount of EDTA, formamide and NaOH.

Based on the above experiments, the applicant has found that the fusion protein Apobec-dCas9 or Apobec-dCpf1 enables site-specifically editing of methylated cytosine site in the target sequence region, which does not rely on the upstream and downstream sequences of the methylated cytosine site, has universal applicability, does not cause off-target effects, and does not introduce other insertion or deletion mutations, so there are no other toxic side effects.

The details will be further described below by way of specific examples. However, it should be understood that the specific embodiments are only used to explain the present invention and are not intended to limit the scope of the present invention. The instruments, devices, reagents, methods and the like used in the present application are all instruments, devices, reagents and methods commonly used in the art unless otherwise specified.

### Examples

### Example 1. Recombinant Protein Expression and Purification

Invitrogen was commissioned to synthesize 6His-NLS-Apobec3H-linker (GGS-GGS-GGS) dCas9(Asp10Ala/His840A1a), 6His-NLS-Apobec3H-linker (GGS-GGS-GGS-GGSGGS-GGS-GGS), 6His-NLS-Apobec3H-linker (GGS-GGS-GGS-GGS-GGS-GGSGGS-GGS-GGS-GGS-GGS-GGS-GGS-GGS)-dCas9(Asp10Ala/His840A1a), 6HisNLS-Apobec3A-linker (GGS-GGS-GGS)-dCas9(Asp10Ala/Hi s840A1a) dCas9(Asp10Ala/His840A1a), 6His-NLS-Apobec3 A-linker (GGS-GGS-GGS-GGSGGS-GGS-GGS)-dCas9(Asp10Ala/His840A1a), 6His-NLS-Apobec3A-linker (GGS-GGS-GGS-GGS-GGS-GGS-GGS-GGS-GGS-GGS-GGS-GGS-GGS-GGS) dCas9(Asp10Ala/His840A1a), 6His-NLS-Apobec3H-linker (GGS-GGS-GGS-GGSGGS-GGS-GGS)-dCpf1(Asp908A1a) gene sequences, respectively SEQ ID NO. 301, NO. 302, NO. 303, NO. 304, NO. 305, NO. 306 and NO. 307, and a Nco I endonuclease site was introduced at the 5′ end of the gene fragment, and a Hind III endonuclease site was introduced at the 3′ end. The synthesized gene fragment and the pET28a (+) vector were respectively double digested with Nco I and Hind III, and the gene fragment and the vector fragment were ligated with T4 DNA ligase, and DH5a competent cells (Tiangen Biochemical Technology (Beijing) Co., Ltd.) were routinely transformed, and positive clones were selected according to kanamycin resistance, then the plasmids were extracted. The recombinant plasmid was identified by Nco I and Hind III double digestion and agarose gel electrophoresis. Meanwhile, Invitrogen was commissioned to sequence the recombinant plasmid, and the results of the sequencing were analyzed using BioEdit software. The results were identical to the designed sequence, indicating that the recombinant plasmid was successfully constructed.

The obtained positive clone plasmid was transformed into E. coli. BL21 (DE3) competent cells (Tiangen Biotechnology (Beijing) Co., Ltd.), and cultured overnight at 37° C. in LB medium containing 100 μg/mlkanamycin, and then transferred to 1 L of the same LB medium and cultured at 37° C. to OD=0.6 about. The medium was then cooled to 4° C. and induced to express for approximately 16 hours by the addition of 0.5 mM IPTG. The cells were collected by centrifugation at 4000 g and resuspended in lysis buffer (50 mM Tris pH=7.0, 1 M NaCl, 20% glycerol, 10 mM TCEP). The cells were lysed by ultrasonic method (6W output for 8 minutes, on for 20 seconds and off for 20 seconds), and the supernatant was separated by centrifugation at 25,000 g. The supernatant was incubated with Nickel resin (ThermoFisher) at 4° C. for 1 hour, then passed through a gravity column and washed with 40 ml of lysis buffer. The recombinant protein was eluted with a 285 mM lysis buffer, diluted to 0.1 M NaCl and concentrated to the appropriate concentration with a centrifuge tube. The quality and concentration of the recombinant protein were determined by SDS Page.

The recombinant protein sequences were SEQ ID NO. 201-207.

### Example 2. sgRNA In Vitro Transcription

Based on the 34 dsDNA substrate sequences to be tested (SEQ ID NO. 39-54 and their complementary strands 55-70, 71-85 and their complementary strands 86-100, 101-104 and their complementary strands 105-108) and the pFYF320 vector sequence providing the sgRNA universal sequence, the sgRNA forward primer (SEQ ID NO. 2-17, 18-34, and 35-38) and the reverse primer (SEQ ID NO. 1) were respectively designed. The sgRNA was obtained from a linear DNA fragment containing the T7 promoter by TranscriptAid T7 High Yield Transcription Kit (ThermoFisher Scientific), using DpnI to remove the template DNA, and then purified using a MEGAclear Kit (ThermoFisher Scientific), and the mass was detected by UV absorption.

### Example 3. Substrate Preparation

Invitrogen was commissioned to synthesize the forward and reverse oligonucleic acid strand sequences of the substrate sequence, wherein the 5′ end of the positive strand sequence was labeled with FAM fluorescent labeling. 2 OD single-stranded oligonucleic acid strands were separately dissolved in 500 μl of water, and an equal amount of the positive and negative chain solutions were mixed and allowed to stand for 5 minutes to obtain a double-stranded substrate (dsDNA).

Fifteen sequences as SEQ ID NO. 39-54 were used for the dCas9 fusion protein demethylation range test.

Fifteen sequences as SEQ ID NO. 71-85 were used for the dCas9 fusion protein demethylation range test.

Four sequences as SEQ ID NO. 101-104 were used to test the effect of the base located adjacent to upstream of the target site on activity.

### Example 4. In Vitro Activity Test

The recombinant fusion protein obtained in Example 1 was separately mixed with the sgRNA obtained in Example 2 in a molar ratio of 1:1, and allowed to stand at room temperature for 5 minutes. The corresponding dsDNA substrate was added to a final concentration of 125 nM and reacted at 37° C. for 2 hours. After the obtained dsDNA was purified using EconoSpin micro spin column (Epoch Life Science), 1 unit of TDG (NEB) was added and reacted at 37° C. for 1 hour. After the reaction, 10 μl of formamide, 1 μl of 0.5 M EDTA, and 0.5 μl of 5 M NaOH were added, and the mixture was reacted at 95° C. for 5 minutes. The product was isolated on 10% TBE-urea gel.

The target DNA strand contained the target Met-C and the 3′ end was labeled with the fluorophore FAM. Under the action of the recombinant protein, Met-C was converted to T and thus could not be paired with G of the complementary strand. Under the action of TDG, the mismatched T was going to be excised, leaving a base deletion site. Under the action of formamide and NaOH, the double strand became a single strand and was further cleaved at the base deletion site, thereby forming a short strand labeled with a fluorescent group FAM. The long and short chain marked DNAs were separated in urea gel. If a long and a short band appeared on the gel, it indicated that the recombinant protein was active.

### Example 5. Preparation of dsDNA Substrate for Pyrosequencing

Invitrogen was commissioned to synthesize the forward and reverse oligonucleic acid strand sequences of the substrate sequence, wherein the 5′ end of the positive strand sequence was labeled with FAM fluorescent labeling. 2 OD single-stranded oligonucleic acid strands were separately dissolved in 500 μl of water, and an equal amount of the positive and negative chain solutions were mixed and allowed to stand for 5 minutes to obtain a double-stranded substrate (dsDNA). The recombinant fusion protein obtained in Example 1 was separately mixed with the sgRNA obtained in Example 2 in a molar ratio of 1:1, and allowed to stand at room temperature for 5 minutes. The corresponding dsDNA substrate was added to a final concentration of 125 nM and reacted at 37° C. for 2 hours. The reacted dsDNA was purified using EconoSpin micro spin column (Epoch Life Science) and submitted to BGI for pyrosequencing after sulfite treatment and amplication with designed primers.

### Example 6. In Vivo Activity Assay

(1) Cell culture

The HEK293 cell line or PC3 cell line was maintained in Dulbecco's Modified Eagle's Medium plus under an environment of 37° C. and 5% carbon dioxide.

(2) Construction of PX330 recombinant protein expression vector

Invitrogen was commissioned to synthesize 6His-NLS-Apobec3H-linker (GGS-GGS-GGS) dCas9(Asp10A1a/His840Ala), 6His-NLS-Apobec3H-linker (GGS-GGS-GGSGGS-GGS-GGS-GGS), 6His-NLS-Apobec3H-linker (GGS-GGS-GGS-GGSGGS-GGS-GGS-GGS-GGS-GGS-GGS-GGS-GGS-GGS) dCas9(Asp10Ala/His840A1a), 6His-NLS-Apobec3A-linker (GGS-GGS-GGS) dCas9(Asp10Ala/His840A1a)-dCas9(Asp10Ala/His840Ala), 6His-NLSApobec3A-linker (GGS-GGS-GGS-GGS-GGS-GGS-GGS) dCas9(Asp10Ala/His840A1a), 6His-NLS-Apobec3A-linker (GGS-GGS-GGSGGS-GGS-GGS-GGS-GGS-GGS-GGS-GGS-GGS-GGS-GGS) dCas9(Asp10A1a/His840Ala), 6His-NLS-Apobec3H-linker (GGS-GGS-GGSGGS-GGS-GGS-GGS)-dCpf1(Asp908A1a) gene sequences, respectively SEQ ID NO. 301, NO. 302, NO. 303, NO. 304, NO. 305, NO. 306 and NO. 307, and a BamHI endonuclease site was introduced at the 5′ end of the gene fragment, and an AgeI endonuclease site was introduced at the 3′ end. The synthesized gene fragment and the pX330 vector (Addgene) were respectively double digested with BamHI and AgeI, and the gene fragment and the vector fragment were ligated with T4 DNA ligase. It was confirmed by sequencing that the recombinant vector was constructed correctly. The sgRNA vectors corresponding to the five intracellular experiments inserted the corresponding PCR products (obtained by PCR from forward primers 121, 123, 125, 127, 129 and reverse primers 1, 122, 124, 126, 128, 130) through MluI and SpeI double digestion.

(3) Transfection

A. One day before transfection, HEK293 cells or PC3 cells were inoculated in a medium that did not contain antibiotics, and the confluence of the cells at the time of transfection was 30-50%.

B. Preparation of transfection samples:

1 μl of 20 μM pX330 recombinant vector and 1.5 μl of cell transfection reagent Lipofectamine™ 2000 (Invitrogen) were diluted in 0.05 ml Opti-MEM (Invitogen), gently mixed and incubated for 5 minutes. The control group was a blank pX330 vector that did not clone any foreign gene.

The diluted pX330 recombinant vector and Lipofectamine™ 2000 (Invitrogen) were incubated at room temperature for 20 minutes to form a recombinant vector-Lipofectamine™ 2000 (Invitrogen) complex and a blank vector-Lipofectamine 2000 (Invitrogen) complex. The incubation time should not exceed 30 minutes, and a longer incubation time may reduce activity.

The vector-Lipofectamine™ 2000 complex was added to each well containing cells and medium, and the plate was gently shaken back and forth, and incubated at 37° C. in a CO₂ incubator for 72 hours.

The transfected cells were harvested 3 days later and the genomic DNA was purified by Agencourt DNA dvance Genomic DNA Isolation Kit (Beckman Coulter). Sample preparation was carried out by the method of Example 5, and the obtained sample was subjected to pyrosequencing by BGI Shenzhen.

### Example 7. Determination of Demethylation Site Range

According to Example 2, the inventor synthesized 30 ssDNA (15 fusion proteins for dCas9, 15 fusion proteins for dCpf1) of 59 bases in length as reaction substrates, their complementary ssDNA, and corresponding sgRNA primers. The 5′ end of the reaction substrate ssDNA was modified by the fluorophore FAM with a methylated C (Met-C) in between, which is the target of editing. After the ssDNA formed a dsDNA substrate with its complementary strand, the Cas9 region of the recombinant protein bound to the corresponding region in the middle of the dsDNA under the guidance of the corresponding sgRNA, and melted about 20 bases in the region, that was, formed a single-stranded region in the middle of the dsDNA. The target Met-C was in this region and was named as substrate 4-20 based on its distance to the 5′-end double-stranded region (4-20 bases). When the recombinant protein bound to different sgRNAs and then interacted with the corresponding dsDNA substrates for a certain period of time, some of the target Met-C became T under deamination and did not pair with G on the complementary strand to form a protrusion. The addition of 1 Unit of TDG after termination of the reaction at high temperature removed the mismatched T base, resulting in a deletion at the editing target of the substrate. The dsDNA then changed back to ssDNA and was cleaved at the base deletion site by the combined action of EDTA (0.5 μl at a concentration of 0.5 M), formamide (10 μl) and NaOH (1 μl at 5 M). Since both the cleaved 5′-end short-chain ssDNA and the unacting ssDNA substrate had a specific FAM fluorophore label at the 5′ end, the relative ratio of the two could be accurately estimated, and the efficiency of the recombinant protein to change Met-C to T at this site could be inferred.

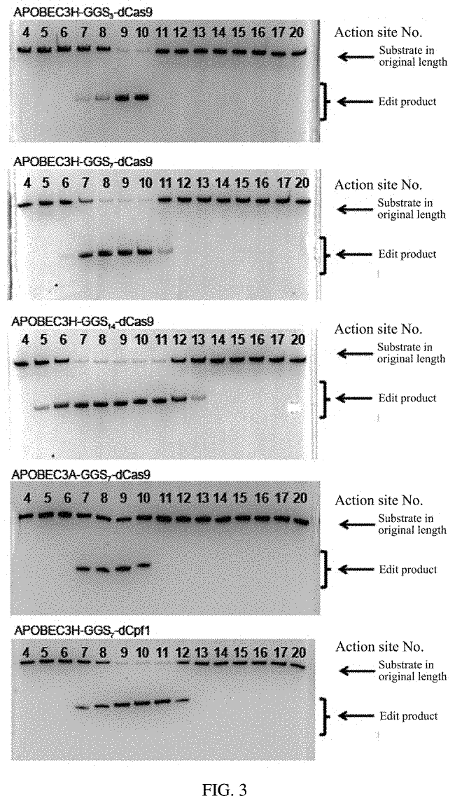

As shown in FIG. 3, by experimental results on 15 different substrates, it can be seen that for the dCas9 fusion protein with a linker of (GGS) 3, Met-C within a range of 7-10 bases from the first base at the 5′ end of the single-stranded region after melting the double-strand in the target region can be changed to T, but not outside the range; for fusion proteins with a linker (GGS) 7 and (GGS) 14, the distances of the editing interval are 6-11 bases and 5-13 bases. This range will be slightly wider due to the length of the linker becoming longer. This range will be an important basis for our subsequent experimental design and future gene therapy design sgRNA.

It can also be seen from the results that A3H was slightly more active than A3A.

As can be seen from the results, the dCpf1 fusion protein with a linker of (GGS) 7 in length had similar activity, and the distance of the action range was 7-12 bases.

In the control group, the synthesized T was used as a positive control, and the wrong sgRNA and Cas-9 or Cpf1 without sgRNA were used as negative controls.

The control experiment was mainly to prove two problems: first, our method is feasible. One of the groups in which the formation of short-chain DNA were clearly seen was chosen, the same ssDNA substrate was synthesized but the Met-C therein was changed to T, that was, the function of the recombinant protein was artificially completed. The same operations were employed. As a result, the formation of short-chain DNA was also observed. It was proved that the short-chain DNA in the experimental results was actually produced by the action of the recombinant protein on the target DNA. Second, by continuing the next experimental procedure by allowing the recombinant protein not to bind to sgRNA or to bind to unpaired sgRNA, no short-chain DNA was produced, demonstrating that such editing was directed.

### Example 8. Effect of Bases Upstream and Downstream of the Action Site

A recombinant protein (a linker of GGS*7, and Apobec protein of A3H) was used as a subject for the study on effect of the base located adjacent to upstream of the editing target site on demethylation activity.

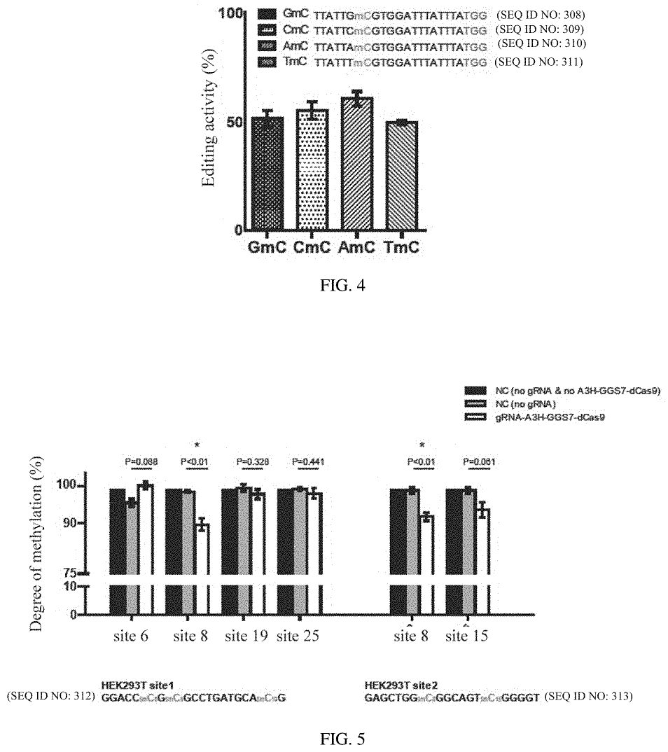

Based on previous studies of the Apobec protein family, the base located adjacent to upstream of the editing target site has a direct effect on their activities. The substrate with Met-C at position 7 was selected and the previous base was changed to A, T, C and G, respectively. As shown in FIG. 4, the test results show that the sequence of the previous base has no effect on the editing efficiency, which proves the versatility of the technology.

### Example 9. Efficiency of Intracellular Demethylation

When it had been demonstrated that the recombinant protein had an ideal ability to change Met-C to T outside the cell, it was desirable to further verify whether such activity remains in the cell, the intensity of the activity, and whether T is repaired into a normal C by the cell's own DNA repair mechanism after the reaction, thereby achieving the effect of site-specific demethylation. The applicant designed three sets of intracellular experiments, and the promoter regions of three different genes were selected for demethylation testing.

The first intracellular editing target was the two methylated C of the U.S. Pat. Nos. 17,741,472 and 17,741,474 loci on chromosome 11 in the HEK293 cell line, located in the promoter region of the gene MYOD1. As shown in FIG. 5, this experiment demonstrated that the system could accurately edit the chosen one in two methylation modifications that were close to each other.

The second editing target was a methylated C of the 31138558 locus on chromosome 6 in the HEK293 cell line, located in the promoter region of the gene POUF1. As shown in FIG. 5, this experiment also achieved the desired editing effect.

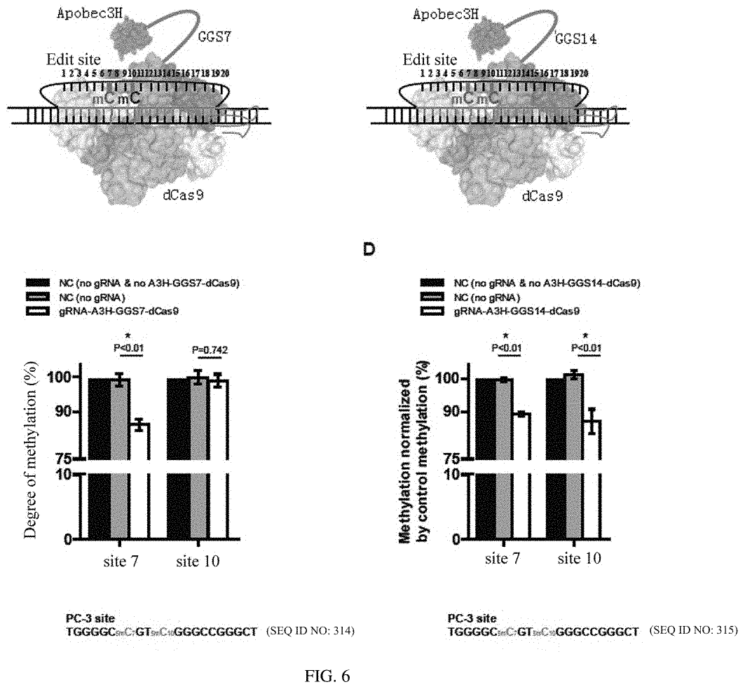

The third editing target was a methylated C of the 113875226 locus on chromosome 2 in the PC3 cell line, located in the promoter region of the gene IL1RN. As shown in FIG. 6, the system can edit one or two of the two adjacent methylated sites by a reasonable sgRNA design.

Recombinant vectors were separately constructed and transfected into cells using the method described in Example 6, and the editing results were evaluated by pyrosequencing.

### Example 10. Proportion of Indel (Insertion and Deletion) in Cells after Editing

Based on the sequencing results of the above experiments, the cases of base insertion and deletion occurring near the target site throughout the process were also counted. From the sequencing results, there was no phenomenon of insertion and deletion of bases around.

The nucleic acid sequences used in the examples are specifically shown in the following table.

| Seq | | |
|---|---|---|
| ID | | |
| no. | Name | Sequence (5′-3′) |
| 1 | Rev_sgRNA_T7 | AAAAAAAGCACCGACTCGGTG |
| 2 | Fwd_sgRNA_T7_ds | TAATACGACTCACTATAGGTATCGGATTTATTTATTTAAGTTT |
| | DNA_4 | TAGAGCTAGAAATAGC |
| 3 | Fwd_sgRNA_T7_ds | TAATACGACTCACTATAGGTTATCGGATTTATTTATTTAGTTT |
| | DNA_5 | TAGAGCTAGAAATAGC |
| 4 | Fwd_sgRNA_T7_ds | TAATACGACTCACTATAGGTTTATCGGATTTATTTATTAGTTT |
| | DNA6 | TAGAGCTAGAAATAGC |
| 5 | Fwd_sgRNA_T7_ds | TAATACGACTCACTATAGGATTTATCGGATTTATTTATTGTTT |
| | DNA_7 | TAGAGCTAGAAATAGC |
| 6 | Fwd_sgRNA_T7_ds | TAATACGACTCACTATAGGTATTTATCGGATTTATTTATGTTT |
| | DNA_8 | TAGAGCTAGAAATAGC |
| 7 | Fwd_sgRNA_T7_ds | TAATACGACTCACTATAGGTTATTTATCGGATTTATTTAGTTT |
| | DNA_9 | TAGAGCTAGAAATAGC |
| 8 | Fwd_sgRNA_T7_ds | TAATACGACTCACTATAGGATTATTTATCGGATTTATTTGTTT |
| | DNA_10 | TAGAGCTAGAAATAGC |
| 9 | Fwd_sgRNA_T7_ds | TAATACGACTCACTATAGGTATTATTTATCGGATTTATTGTTT |
| | DNA_11 | TAGAGCTAGAAATAGC |
| 10 | Fwd_sgRNA_T7_ds | TAATACGACTCACTATAGGATTATTATTATCGGATTTATGTTT |
| | DNA_12 | TAGAGCTAGAAATAGC |
| 11 | Fwd_sgRNA_T7_ds | TAATACGACTCACTATAGGTATTATATTTATCGGATTTAGTTT |
| | DNA_13 | TAGAGCTAGAAATAGC |
| 12 | Fwd_sgRNA_T7_ds | TAATACGACTCACTATAGGTTATTATATTTATCGGATTTGTTT |
| | DNA_14 | TAGAGCTAGAAATAGC |
| 13 | Fwd_sgRNA_T7_ds | TAATACGACTCACTATAGGATTATTATATTTATCGGATTGTTT |
| | DNA_15 | TAGAGCTAGAAATAGC |
| 14 | Fwd_sgRNA_T7_ds | TAATACGACTCACTATAGGTATTATTATATTTATCGGATGTTT |
| | DNA_16 | TAGAGCTAGAAATAGC |
| 15 | Fwd_sgRNA_T7_ds | TAATACGACTCACTATAGGATTATTATTATTATATCGGAGTTT |
| | DNA_17 | TAGAGCTAGAAATAGC |
| 16 | Fwd_sgRNA_T7_ds | TAATACGACTCACTATAGGATTATTATTATTATTATATCGTTT |
| | DNA_20 | TAGAGCTAGAAATAGC |
| 17 | Fwd_sgRNA_T7_ds | TAATACGACTCACTATAGGTATAGGATTTATTTATTTAAGTTT |
| | DNA_noC | TAGAGCTAGAAATAGC |
| 18 | Fwd_crRNA_T7 | TAATACGACTCACTATAGGAATTTCTACTGTTGTAGATG |
| 19 | Rev_crRNA_T7_dsD | TTAAATAAATAAATCCGATACATCTACAACAGTAGAAATTCC |
| | NA_4 | TATAGTGAGTCGTATTA |
| 20 | Rev_crRNA_T7_dsD | TAAATAAATAAATCCGATAACATCTACAACAGTAGAAATTCC |
| | NA_5 | TATAGTGAGTCGTATTA |
| 21 | Rev_crRNA_T7_dsD | TAATAAATAAATCCGATAAACATCTACAACAGTAGAAATTCC |
| | NA_6 | TATAGTGAGTCGTATTA |
| 22 | Rev_crRNA_T7_dsD | AATAAATAAATCCGATAAATCATCTACAACAGTAGAAATTCC |
| | NA_7 | TATAGTGAGTCGTATTA |
| 23 | Rev_crRNA_T7_dsD | ATAAATAAATCCGATAAATACATCTACAACAGTAGAAATTCC |
| | NA_8 | TATAGTGAGTCGTATTA |
| 24 | Rev_crRNA_T7_dsD | TAAATAAATCCGATAAATAACATCTACAACAGTAGAAATTCC |
| | NA_9 | TATAGTGAGTCGTATTA |
| 25 | Rev_crRNA_T7_dsD | AAATAAATCCGATAAATAATCATCTACAACAGTAGAAATTCC |
| | NA_10 | TATAGTGAGTCGTATTA |
| 26 | Rev_crRNA_T7_dsD | AATAAATCCGATAAATAATACATCTACAACAGTAGAAATTCC |
| | NA_11 | TATAGTGAGTCGTATTA |
| 27 | Rev_crRNA_T7_dsD | ATAAATCCGATAATAATAATCATCTACAACAGTAGAAATTCC |
| | NA_12 | TATAGTGAGTCGTATTA |
| 28 | Rev_crRNA_T7_dsD | TAAATCCGATAAATATAATACATCTACAACAGTAGAAATTCC |
| | NA_13 | TATAGTGAGTCGTATTA |
| 29 | Rev_crRNA_T7_dsD | AAATCCGATAAATATAATAACATCTACAACAGTAGAAATTCC |
| | NA_14 | TATAGTGAGTCGTATTA |
| 30 | Rev_crRNA_T7_dsD | AATCCGATAAATATAATAATCATCTACAACAGTAGAAATTCC |
| | NA_15 | TATAGTGAGTCGTATTA |
| 31 | Rev_crRNA_T7_dsD | ATCCGATAAATATAATAATACATCTACAACAGTAGAAATTCC |
| | NA_16 | TATAGTGAGTCGTATTA |
| 32 | Rev_crRNA_T7_dsD | TCCGATATAATAATAATAATCATCTACAACAGTAGAAATTCC |
| | NA_17 | TATAGTGAGTCGTATTA |
| 33 | Rev_crRNA_T7_dsD | GATATAATAATAATAATAATCATCTACAACAGTAGAAATTCC |
| | NA_20 | TATAGTGAGTCGTATTA |
| 34 | Rev_crRNA_T7_dsD | TTAAATAAATAAATCCTATACATCTACAACAGTAGAAATTCC |
| | NA_noC | TATAGTGAGTCGTATTA |
| 35 | Fwd_sgRNA_6T | TAATACGACTCACTATAGGTTATTTCGTGGATTTATTTAGTTT |
| | | TAGAGCTAGAAATAGC |
| 36 | Fwd_sgRNA_6A | TAATACGACTCACTATAGGTTATTTCGTGGATTTATTTAGTTT |
| | | TAGAGCTAGAAATAGC |
| 37 | Fwd_sgRNA_6C | TAATACGACTCACTATAGGTTATTTCGTGGATTTATTTAGTTT |
| | | TAGAGCTAGAAATAGC |
| 38 | Fwd_sgRNA_6G | TAATACGACTCACTATAGGTTATTTCGTGGATTTATTTAGTTT |
| | | TAGAGCTAGAAATAGC |
| 39 | dCas9_ds_4 | FAM- |
| | | GGTAGTTAGGATGAATGGAAGGTTGGTATAGCCTATmet- |
| | | CGGATTTATTTATTTAAT |
| | | GGATGACCTCTGGATCCATG |
| 40 | dCas9_ds_5 | FAM- |
| | | GGTAGTTAGGATGAATGGAAGGTTGGTATAGCCTTATmet- |
| | | CGGATTTATTTATTTAT |
| | | GGATGACCTCTGGATCCATG |
| 41 | dCas9_ds_6 | FAM- |
| | | GGTAGTTAGGATGAATGGAAGGTTGGTATAGCCTTTATmet- |
| | | CGGATTTATTTATTAT |
| | | GGATGACCTCTGGATCCATG |
| 42 | dCas9_ds_7 | FAM- |
| | | GGTAGTTAGGATGAATGGAAGGTTGGTATAGCCATTTATmet- |
| | | CGGATTTATTTATTT |
| | | GGATGACCTCTGGATCCATG |
| 43 | dCas9_ds_8 | FAM- |
| | | GGTAGTTAGGATGAATGGAAGGTTGGTATAGCCTATTTATmet |
| | | -CGGATTTATTTATT |
| | | GGATGACCTCTGGATCCATG |
| 44 | dCas9_ds_9 | FAM- |
| | | GGTAGTTAGGATGAATGGAAGGTTGGTATAGCCTTATTTATm |
| | | et-CGGATTTATTTAT |
| | | GGATGACCTCTGGATCCATG |
| 45 | dCas9_ds_10 | FAM- |
| | | GGTAGTTAGGATGAATGGAAGGTTGGTATAGCCATTATTTAT |
| | | met-CGGATTTATTTT |
| | | GGATGACCTCTGGATCCATG |
| 46 | dCas9_ds_11 | FAM- |
| | | GGTAGTTAGGATGAATGGAAGGTTGGTATAGCCTATTATTTA |
| | | Tmet-CGGATTTATTT |
| | | GGATGACCTCTGGATCCATG |
| 47 | dCas9_ds_12 | FAM- |
| | | GGTAGTTAGGATGAATGGAAGGTTGGTATAGCCATTATTATT |
| | | ATmet-CGGATTTATT |
| | | GGATGACCTCTGGATCCATG |
| 48 | dCas9_ds_13 | FAM- |
| | | GGTAGTTAGGATGAATGGAAGGTTGGTATAGCCTATTATATT |
| | | TATmet-CGGATTTAT |
| | | GGATGACCTCTGGATCCATG |
| 49 | dCas9_ds_14 | FAM- |
| | | GGTAGTTAGGATGAATGGAAGGTTGGTATAGCCTTATTATAT |
| | | TTATmet-CGGATTTT |
| | | GGATGACCTCTGGATCCATG |
| 50 | dCas9_ds_15 | FAM- |
| | | GGTAGTTAGGATGAATGGAAGGTTGGTATAGCCATTATTATA |
| | | TTTATmet-CGGATTT |
| | | GGATGACCTCTGGATCCATG |
| 51 | dCas9_ds_16 | FAM- |
| | | GGTAGTTAGGATGAATGGAAGGTTGGTATAGCCTATTATTAT |
| | | ATTTATmet-CGGATT |
| | | GGATGACCTCTGGATCCATG |
| 52 | dCas9_ds_17 | FAM- |
| | | GGTAGTTAGGATGAATGGAAGGTTGGTATAGCCATTATTATT |
| | | ATTATATmet-CGGAT |
| | | GGATGACCTCTGGATCCATG |
| 53 | dCas9_ds_20 | FAM- |
| | | GGTAGTTAGGATGAATGGAAGGTTGGTATAGCCATTATTATT |
| | | ATTATTATATmet-CT |
| | | GGATGACCTCTGGATCCATG |
| 54 | dCas9_ds_noC | FAM- |
| | | GGTAGTTAGGATGAATGGAAGGTTGGTATAGCCTATAGGATT |
| | | TATTTATTTAAT |
| | | GGATGACCTCTGGATCCATG |
| 55 | dCas9_ds_com_4 | CATGGATCCAGAGGTCATCCATTAAATAAATAAATCCGATAG |
| | | GCTATACCAACCTTCC |
| | | ATTCATCCTAACTACC |
| 56 | dCas9_ds_com_5 | CATGGATCCAGAGGTCATCCATAAATAAATAAATCCGATAA |
| | | GGCTATACCAACCTTCC |
| | | ATTCATCCTAACTACC |
| 57 | dCas9_ds_com_6 | CATGGATCCAGAGGTCATCCATAATAAATAAATCCGATAAA |
| | | GGCTATACCAACCTTCC |
| | | ATTCATCCTAACTACC |
| 58 | dCas9_ds_com_7 | CATGGATCCAGAGGTCATCCAAATAAATAAATCCGATAAAT |
| | | GGCTATACCAACCTTCC |
| | | ATTCATCCTAACTACC |
| 59 | dCas9_ds_com_8 | CATGGATCCAGAGGTCATCCAATAAATAAATCCGATAAATA |
| | | GGCTATACCAACCTTCC |
| | | ATTCATCCTAACTACC |
| 60 | dCas9_ds_com_9 | CATGGATCCAGAGGTCATCCATAAATAAATCCGATAAATAA |
| | | GGCTATACCAACCTTCC |
| | | ATTCATCCTAACTACC |
| 61 | dCas9_ds_com_10 | CATGGATCCAGAGGTCATCCAAAATAAATCCGATAAATAAT |
| | | GGCTATACCAACCTTCC |
| | | ATTCATCCTAACTACC |
| 62 | dCas9_ds_com_11 | CATGGATCCAGAGGTCATCCAAATAAATCCGATAAATAATA |
| | | GGCTATACCAACCTTCC |
| | | ATTCATCCTAACTACC |
| 63 | dCas9_ds_com_12 | CATGGATCCAGAGGTCATCCAATAAATCCGATAATAATAATG |
| | | GCTATACCAACCTTCC |
| | | ATTCATCCTAACTACC |
| 64 | dCas9_ds_com_13 | CATGGATCCAGAGGTCATCCATAAATCCGATAAATATAATAG |
| | | GCTATACCAACCTTCC |
| | | ATTCATCCTAACTACC |
| 65 | dCas9_ds_com_14 | CATGGATCCAGAGGTCATCCAAAATCCGATAAATATAATAA |
| | | GGCTATACCAACCTTCC |
| | | ATTCATCCTAACTACC |
| 66 | dCas9_ds_com_15 | CATGGATCCAGAGGTCATCCAAATCCGATAAATATAATAATG |
| | | GCTATACCAACCTTCC |
| | | ATTCATCCTAACTACC |
| 67 | dCas9_ds_com_16 | CATGGATCCAGAGGTCATCCAATCCGATAAATATAATAATAG |
| | | GCTATACCAACCTTCC |
| | | ATTCATCCTAACTACC |
| 68 | dCas9_ds_com_17 | CATGGATCCAGAGGTCATCCATCCGATATAATAATAATAATG |
| | | GCTATACCAACCTTCC |
| | | ATTCATCCTAACTACC |
| 69 | dCas9_ds_com_20 | CATGGATCCAGAGGTCATCCAGATATAATAATAATAATAATG |
| | | GCTATACCAACCTTCC |
| | | ATTCATCCTAACTACC |
| 70 | dCas9_ds_com_noC | CATGGATCCAGAGGTCATCCATTAAATAAATAAATCCTATAG |
| | | GCTATACCAACCTTCC |
| | | ATTCATCCTAACTACC |
| 71 | dCpf1_ds_4 | FAM-GGTACCCGGGGATCCTTTATATmet- |
| | | CGGATTTATTTATTTAAGTTAAAAAGCTTGGCGTAAT |
| 72 | dCpf1_ds_5 | FAM-GGTACCCGGGGATCCTTTATTATmet- |
| | | CGGATTTATTTATTTAGTTAAAAAGCTTGGCGTAAT |
| 73 | dCpf1_ds_6 | FAM-GGTACCCGGGGATCCTTTATTTATmet- |
| | | CGGATTTATTTATTAGTTAAAAAGCTTGGCGTAAT |
| 74 | dCpf1_ds_7 | FAM-GGTACCCGGGGATCCTTTAATTTATmet- |
| | | CGGATTTATTTATTGTTAAAAAGCTTGGCGTAAT |
| 75 | dCpf1_ds_8 | FAM-GGTACCCGGGGATCCTTTATATTTATmet- |
| | | CGGATTTATTTATGTTAAAAAGCTTGGCGTAAT |
| 76 | dCpf1_ds_9 | FAM-GGTACCCGGGGATCCTTTATTATTTATmet- |
| | | CGGATTTATTTAGTTAAAAAGCTTGGCGTAAT |
| 77 | dCpf1_ds_10 | FAM-GGTACCCGGGGATCCTTTAATTATTTATmet- |
| | | CGGATTTATTTGTTAAAAAGCTTGGCGTAAT |
| 78 | dCpf1_ds_11 | FAM-GGTACCCGGGGATCCTTTATATTATTTATmet- |
| | | CGGATTTATTGTTAAAAAGCTTGGCGTAAT |
| 79 | dCpf1_ds_12 | FAM-GGTACCCGGGGATCCTTTAATTATTATTATmet- |
| | | CGGATTTATGTTAAAAAGCTTGGCGTAAT |
| 80 | dCpf1_ds_13 | FAM-GGTACCCGGGGATCCTTTATATTATATTTATmet- |
| | | CGGATTTAGTTAAAAAGCTTGGCGTAAT |
| 81 | dCpf1_ds_14 | FAM-GGTACCCGGGGATCCTTTATTATTATATTTATmet- |
| | | CGGATTTGTTAAAAAGCTTGGCGTAAT |
| 82 | dCpf1_ds_15 | FAM-GGTACCCGGGGATCCTTTAATTATTATATTTATmet- |
| | | CGGATTGTTAAAAAGCTTGGCGTAAT |
| 83 | dCpf1_ds_16 | FAM-GGTACCCGGGGATCCTTTATATTATTATATTTATmet- |
| | | CGGATGTTAAAAAGCTTGGCGTAAT |
| 84 | dCpf1_ds_17 | FAM-GGTACCCGGGGATCCTTTAATTATTATTATTATATmet- |
| | | CGGAGTTAAAAAGCTTGGCGTAAT |
| 85 | dCpf1_ds_20 | FAM- |
| | | GGTACCCGGGGATCCTTTAATTATTATTATTATTATATmet- |
| | | CGTTAAAAAGCTTGGCGTAAT |
| 86 | dCpf1_ds_com_4 | ATTACGCCAAGCTTTTTAACTTAAATAAATAAATCCGATATA |
| | | AAGGATCCCCGGGTACC |
| 87 | dCpf1_ds_com_5 | ATTACGCCAAGCTTTTTAACTAAATAAATAAATCCGATAATA |
| | | AAGGATCCCCGGGTACC |
| 88 | dCpf1_ds_com_6 | ATTACGCCAAGCTTTTTAACTAATAAATAAATCCGATAAATA |
| | | AAGGATCCCCGGGTACC |
| 89 | dCpf1_ds_com_7 | ATTACGCCAAGCTTTTTAACAATAAATAAATCCGATAAATTA |
| | | AAGGATCCCCGGGTACC |
| 90 | dCpf1_ds_com_8 | ATTACGCCAAGCTTTTTAACATAAATAAATCCGATAAATATA |
| | | AAGGATCCCCGGGTACC |
| 91 | dCpf1_ds_com_9 | ATTACGCCAAGCTTTTTAACTAAATAAATCCGATAAATAATA |
| | | AAGGATCCCCGGGTACC |
| 92 | dCpf1_ds_com_10 | ATTACGCCAAGCTTTTTAACAAATAAATCCGATAAATAATTA |
| | | AAGGATCCCCGGGTACC |
| 93 | dCpf1_ds_com_11 | ATTACGCCAAGCTTTTTAACAATAAATCCGATAAATAATATA |
| | | AAGGATCCCCGGGTACC |
| 94 | dCpf1_ds_com_12 | ATTACGCCAAGCTTTTTAACATAAATCCGATAATAATAATTA |
| | | AAGGATCCCCGGGTACC |
| 95 | dCpf1_ds_com_13 | ATTACGCCAAGCTTTTTAACTAAATCCGATAAATATAATATA |
| | | AAGGATCCCCGGGTACC |
| 96 | dCpf1_ds_com_14 | ATTACGCCAAGCTTTTTAACAAATCCGATAAATATAATAATA |
| | | AAGGATCCCCGGGTACC |
| 97 | dCpf1_ds_com_15 | ATTACGCCAAGCTTTTTAACAATCCGATAAATATAATAATTA |
| | | AAGGATCCCCGGGTACC |
| 98 | dCpf1_ds_com_16 | ATTACGCCAAGCTTTTTAACATCCGATAAATATAATAATATA |
| | | AAGGATCCCCGGGTACC |
| 99 | dCpf1_ds_com_17 | ATTACGCCAAGCTTTTTAACTCCGATATAATAATAATAATTA |
| | | AAGGATCCCCGGGTACC |
| 100 | dCpf1_ds_com_20 | ATTACGCCAAGCTTTTTAACGATATAATAATAATAATAATTA |
| | | AAGGATCCCCGGGTACC |
| 101 | dCas9_ds_6T | ACGTAAACGGCCACAAGTTCTTATTTmet- |
| | | CGTGGATTTATTTATGGCATCTTCTTCAAGGAC |
| 102 | dCas9_ds_6A | ACGTAAACGGCCACAAGTTCTTATTAmet- |
| | | CGTGGATTTATTTATGGCATCTTCTTCAAGGAC |
| 103 | dCas9_ds_6C | ACGTAAACGGCCACAAGTTCTTATTCmet- |
| | | CGTGGATTTATTTATGGCATCTTCTTCAAGGAC |
| 104 | dCas9_ds_6G | ACGTAAACGGCCACAAGTTCTTATTGmet- |
| | | CGTGGATTTATTTATGGCATCTTCTTCAAGGAC |
| 105 | dCas9_ds_com_6T | GTCCTTGAAGAAGATGCCATAAATAAATCCACGAAATAAGA |
| | | ACTTGTGGCCGTTTACGT |
| 106 | dCas9_ds_com_6A | GTCCTTGAAGAAGATGCCATAAATAAATCCACGTAATAAGA |
| | | ACTTGTGGCCGTTTACGT |
| 107 | dCas9_ds_com_6C | GTCCTTGAAGAAGATGCCATAAATAAATCCACGGAATAAGA |
| | | ACTTGTGGCCGTTTACGT |
| 108 | dCas9_ds_com_6G | GTCCTTGAAGAAGATGCCATAAATAAATCCACGCAATAAGA |
| | | ACTTGTGGCCGTTTACGT |
| 109 | ds_6_F | CGTAAACGGCCACAAGTTCTTAT |
| 110 | ds_6_R | GTCCTTGAAGAAGATGCCATAAA |
| 111 | ds_6_S | CGGCCACAAGTTCTTAT |
| 112 | HEK293T-T1-F | GGATTTGYGTTTTTTYGAAGATTTGG |
| 113 | HEK293T-T1-R | AAATACRAATACTCTTCRAATTTCAAAAAC |
| 114 | HEK293T-T1-S | GTTTTTTAGAAGATTTGGAT |
| 115 | HEK293T-T2-F | GTTTTGAATGAATGTGTGTATATATGTATG |
| 116 | HEK293T-T2-R | CTAACAAAAACCAAACTAATTCTTATCTAC |
| 117 | HEK293T-T2-S | ATGAATGTGTGTATATATGTATGAG |
| 118 | PC3-F | TAAGGGTTTTYGGAAYGGGGT |
| 119 | PC3-R | CCAAACAAAACATCCCTCAAC |
| 120 | PC3-S | GGGTTGTGTGAGTGGG |
| 121 | HEK293T-gRNA1-F | CACCG GGACCCGCGCCTGATGCACG |
| 122 | HEK293T-gRNA1-R | AAAC CGTGCATCAGGCGCGGGTCC C |
| 123 | HEK293T-gRNA2-F | CACCG GAGCTGGCGGCAGTCGGGGT |
| 124 | HEK293T-gRNA2-R | AAAC ACCCCGACTGCCGCCAGCTC C |
| 125 | Gfap-gRNA-F | CACCG TTCCGAGAAGTCTATTGAGC |
| 126 | Gfap-gRNA-R | AAAC GCTCAATAGACTTCTCGGAA C |
| 127 | PMP24-gRNA-F | CACCG TGGGGCCGTCGGGCCGGGCT |
| 128 | PMP24-gRNA-R | AAAC AGCCCGGCCCGACGGCCCCA C |
| 129 | C/EBPδ-gRNA-F | CACCG TCAGCCGGGGCTAGAAAAGG |

The sequences of protein domains are as follows:

APOBEC3A
MEASPASGPRHLMDPHIFTSNFNNGIGRHKTYLCYEVERL
DNGTSVKMDQHRGFLHNQAKNLLCGFYGRHAELRFLDLVP
SLQLDPAQIYRVTWFISWSPCFSWGCAGEVRAFLQENTHV
RLRIFAARIYDYDPLYKEALQMLRDAGAQVSIMTYDEFKH
CWDTFVDHQGCPFQPWDGLDEHSQALSGRLRAILQNQGN
>AP0BEC3H Hyplotype II
MALLTAETFRLQFNNKRRLRRPYYPRKALLCYQLTPQNGS
TPTRGYFENKKKCHAEICFINEIKSMGLDETQCYQVTCYL
TWSPCSSCAWELVDFIKAHDHLNLRIFASRLYYHWCKPQQ
DGLRLLCGSQVPVEVMGFPEFADCWENFVDHEKPLSFNPY
KMLEELDKNSRAIKRRLDRIKS
>Cas9
MDKKYSIGLDIGTNSVGWAVITDEYKVPSKKFKVLGNTDR
HSIKKNLIGALLFDSGETAEATRLKRTARRRYTRRKNRIC
YLQEIFSNEMAKVDDSFFHRLEESFLVEEDKKHERHPIFG
NIVDEVAYHEKYPTIYHLRKKLVDSTDKADLRLIYLALAH
MIKFRGHFLIEGDLNPDNSDVDKLFIQLVQTYNQLFEENP
INASGVDAKAILSARLSKSRRLENLIAQLPGEKKNGLFGN
LIALSLGLTPNFKSNFDLAEDAKLQLSKDTYDDDLDNLLA
QIGDQYADLFLAAKNLSDAILLSDILRVNTEITKAPLSAS
MIKRYDEHHQDLTLLKALVRQQLPEKYKEIFFDQSKNGYA
GYIDGGASQEEFYKFIKPILEKMDGTEELLVKLNREDLLR
KQRTFDNGSIPHQIHLGELHAILRRQEDFYPFLKDNREKI
EKILTFRIPYYVGPLARGNSRFAWMTRKSEETITPWNFEE
VVDKGASAQSFIERMTNFDKNLPNEKVLPKHSLLYEYFTV
YNELTKVKYVTEGMRKPAFLSGEQKKAIVDLLFKTNRKVT
VKQLKEDYFKKIECFDSVEISGVEDRFNASLGTYHDLLKI
IKDKDFLDNEENEDILEDIVLTLTLFEDREMIEERLKTYA
HLFDDKVMKQLKRRRYTGWGRLSRKLINGIRDKQSGKTIL
DFLKSDGFANRNFMQLIHDDSLTFKEDIQKAQVSGQGDSL
HEHIANLAGSPAIKKGILQTVKVVDELVKVMGRHKPENIV
IEMARENQTTQKGQKNSRERMKRIEEGIKELGSQILKEHP
VENTQLQNEKLYEYYLQNGRDMYVDQELDINRLSDYDVDH
IVPQSFLKDDSIDNKVLTRSDKNRGKSDNVPSEEVVKKMK
NYWRQLLNAKLITQRKFDNLTKAERGGLSELDKAGFIKRQ
LVETRQITKHVAQILDSRMNTKYDENDKLIREVKVITLKS
KLVSDFRKDFQFYKVREINNYHHAHDAYLNAVVGTALIKK
YPKLESEFVYGDYKVYDVRKMIAKSEQEIGKATAKYFFYS
NIMNFFKTEITLANGEIRKRPLIETNGETGEIVWDKGRDF
ATVRKVLSMPQVNIVKKTEVQTGGFSKESILPKRNSDKLI
ARKKDWDPKKYGGFDSPTVAYSVLVVAKVEKGKSKKLKSV
KELLGITIMERSSFEKNPIDFLEAKGYKEVKKDLIIKLPK
YSLFELENGRKRMLASAGELQKGNELALPSKYVNFLYLAS
HYEKLKGSPEDNEQKQLFVEQHKHYLDEIIEQISEFSKRV
ILADANLDKVLSAYNKHRDKPIREQAENIIHLFTLTNLGA
PAAFKYFDTTIDRKRYTSTKEVLDATLIHQSITGLYETRI
DLSQLGGDPPKKKRKV
>Cpf1
MTQFEGFTNLYQVSKTLRFELIPQGKTLKHIQEQGFIEED
KARNDHYKELKPIIDRIYKTYADQCLQLVQLDWENLSAAI
DSYRKEKTEETRNALIEEQATYRNAIHDYFIGRTDNLTDA
INKRHAEIYKGLFKAELFNGKVLKQLGTVTTTEHENALLR
SFDKFTTYFSGFYENRKNVFSAEDISTAIPHRIVQDNFPK
FKENCHIFTRLITAVPSLREHFENVKKAIGIFVSTSIEEV
FSFPFYNQLLTQTQIDLYNQLLGGISREAGTEKIKGLNEV
LNLAIQKNDETAHIIASLPHRFIPLFKQILSDRNTLSFIL
EEFKSDEEVIQSFCKYKTLLRNENVLETAEALFNELNSID
LTHIFISHKKLETISSALCDHWDTLRNALYERRISELTGK
ITKSAKEKVQRSLKHEDINLQEIISAAGKELSEAFKQKTS
EILSHAHAALDQPLPTTLKKQEEKEILKSQLDSLLGLYHL
LDWFAVDESNEVDPEFSARLTGIKLEMEPSLSFYNKARNY
ATKKPYSVEKFKLNFQMPTLASGWDVNKEKNNGAILFVKN
GLYYLGIMPKQKGRYKALSFEPTEKTSEGFDKMYYDYFPD
AAKMIPKCSTQLKAVTAHFQTHTTPILLSNNFIEPLEITK
EIYDLNNPEKEPKKFQTAYAKKTGDQKGYREALCKWIDFT
RDFLSKYTKTTSIDLSSLRPSSQYKDLGEYYAELNPLLYH
ISFQRIAEKEIMDAVETGKLYLFQIYNKDFAKGHHGKPNL
HTLYWTGLFSPENLAKTSIKLNGQAELFYRPKSRMKRMAH
RLGEKMLNKKLKDQKTPIPDTLYQELYDYVNHRLSHDLSD
EARALLPNVITKEVSHEIIKDRRFTSDKFFFHVPITLNYQ
AANSPSKFNQRVNAYLKEHPETPIIGIDRGERNLIYITVI
DSTGKILEQRSLNTIQQFDYQKKLDNREKERVAARQAWSV
VGTIKDLKQGYLSQVIHEIVDLMIHYQAVVVLENLNFGFK
SKRTGIAEKAVYQQFEKMLIDKLNCLVLKDYPAEKVGGVL
NPYQLTDQFTSFAKMGTQSGFLFYVPAPYTSKIDPLTGFV
DPFVWKTIKNHESRKHFLEGFDFLHYDVKTGDFILHFKMN
RNLSFQRGLPGFMPAWDIVFEKNETQFDAKGTPFIAGKRI
VPVIENHRFTGRYRDLYPANELIALLEEKGIVFRDGSNIL
PKLLENDDSHAIDTMVALIRSVLQMRNSNAATGEDYINSP
VRDLNGVCFDSRFQNPEWPMDADANGAYHIALKGQLLLNH
LKESKDLKLQNGISNQDWLAYIQELRN
Seq ID NO 201:
>6his-NLS-A3A-GGS3-dCas9
HHHHHH-SSGLVPRGSHM-PKKKRKV-MEASPASGPRHLM
DPHIFTSNFNNGIGRHKTYLCYEVERLDNGTSVKMDQHRG
FLHNQAKNLLCGFYGRHAELRFLDLVPSLQLDPAQIYRVT
WFISWSPCFSWGCAGEVRAFLQENTHVRLRIFAARIYDYD
PLYKEALQMLRDAGAQVSIMTYDEFKHCWDTFVDHQGCPF
QPWDGLDEHSQALSGRLRAILQNQGN-GGSGGSGGS-MDK
KYSIGLAIGTNSVGWAVITDEYKVPSKKFKVLGNTDRHSI
KKNLIGALLFDSGETAEATRLKRTARRRYTRRKNRICYLQ
EIFSNEMAKVDDSFFHRLEESFLVEEDKKHERHPIFGNIV
DEVAYHEKYPTIYHLRKKLVDSTDKADLRLIYLALAHMIK
FRGHFLIEGDLNPDNSDVDKLFIQLVQTYNQLFEENPINA
SGVDAKAILSARLSKSRRLENLIAQLPGEKKNGLFGNLIA
LSLGLTPNFKSNFDLAEDAKLQLSKDTYDDDLDNLLAQIG
DQYADLFLAAKNLSDAILLSDILRVNTEITKAPLSASMIK
RYDEHHQDLTLLKALVRQQLPEKYKEIFFDQSKNGYAGYI
DGGASQEEFYKFIKPILEKMDGTEELLVKLNREDLLRKQR
TFDNGSIPHQIHLGELHAILRRQEDFYPFLKDNREKIEKI
LTFRIPYYVGPLARGNSRFAWMTRKSEETITPWNFEEVVD
KGASAQSFIERMTNFDKNLPNEKVLPKHSLLYEYFTVYNE
LTKVKYVTEGMRKPAFLSGEQKKAIVDLLFKTNRKVTVKQ
LKEDYFKKIECFDSVEISGVEDRFNASLGTYHDLLKIIKD
KDFLDNEENEDILEDIVLTLTLFEDREMIEERLKTYAHLF
DDKVMKQLKRRRYTGWGRLSRKLINGIRDKQSGKTILDFL
KSDGFANRNFMQLIHDDSLTFKEDIQKAQVSGQGDSLHEH
IANLAGSPAIKKGILQTVKVVDELVKVMGRHKPENIVIEM
ARENQTTQKGQKNSRERMKRIEEGIKELGSQILKEHPVEN
TQLQNEKLYLYYLQNGRDMYVDQELDINRLSDYDVDAIVP
QSFLKDDSIDNKVLTRSDKNRGKSDNVPSEEVVKKMKNYW
RQLLNAKLITQRKFDNLTKAERGGLSELDKAGFIKRQLVE
TRQITKHVAQILDSRMNTKYDENDKLIREVKVITLKSKLV
SDFRKDFQFYKVREINNYHHAHDAYLNAVVGTALIKKYPK
LESEFVYGDYKVYDVRKMIAKSEQEIGKATAKYFFYSNIM
NFFKTEITLANGEIRKRPLIETNGETGEIVWDKGRDFATV
RKVLSMPQVNIVKKTEVQTGGFSKESILPKRNSDKLIARK
KDWDPKKYGGFDSPTVAYSVLVVAKVEKGKSKKLKSVKEL
LGITIMERSSFEKNPIDFLEAKGYKEVKKDLIIKLPKYSL
FELENGRKRMLASAGELQKGNELALPSKYVNFLYLASHYE
KLKGSPEDNEQKQLFVEQHKHYLDEIIEQISEFSKRVILA
DANLDKVLSAYNKHRDKPIREQAENIIHLFTLTNLGAPAA
FKYFDTTIDRKRYTSTKEVLDATLIHQSITGLYETRIDLS
QLGGDPPKKKRKV
Seq ID NO 202:
>6his-NLS-A3A-GGS7-dCas9
HHHHHH-SSGLVPRGSHM-PKKKRKV-EASPASGPRHLMD
PHIFTSNFNNGIGRHKTYLCYEVERLDNGTSVKMDQHRGF
LHNQAKNLLCGFYGRHAELRFLDLVPSLQLDPAQIYRVTW
FISWSPCFSWGCAGEVRAFLQENTHVRLRIFAARIYDYDP
LYKEALQMLRDAGAQVSIMTYDEFKHCWDTFVDHQGCPFQ
PWDGLDEHSQALSGRLRAILQNQGN-GGSGGSGGSGGSGG
SGGSGGS-MDKKYSIGLAIGTNSVGWAVITDEYKVPSKKF
KVLGNTDRHSIKKNLIGALLFDSGETAEATRLKRTARRRY
TRRKNRICYLQEIFSNEMAKVDDSFFHRLEESFLVEEDKK
HERHPIFGNIVDEVAYHEKYPTIYHLRKKLVDSTDKADLR
LIYLALAHMIKFRGHFLIEGDLNPDNSDVDKLFIQLVQTY
NQLFEENPINASGVDAKAILSARLSKSRRLENLIAQLPGE
KKNGLFGNLIALSLGLTPNFKSNFDLAEDAKLQLSKDTYD
DDLDNLLAQIGDQYADLFLAAKNLSDAILLSDILRVNTEI
TKAPLSASMIKRYDEHHQDLTLLKALVRQQLPEKYKEIFF
DQSKNGYAGYIDGGASQEEFYKFIKPILEKMDGTEELLVK
LNREDLLRKQRTFDNGSIPHQIHLGELHAILRRQEDFYPF
LKDNREKIEKILTFRIPYYVGPLARGNSRFAWMTRKSEET
ITPWNFEEVVDKGASAQSFIERMTNFDKNLPNEKVLPKHS
LLYEYFTVYNELTKVKYVTEGMRKPAFLSGEQKKAIVDLL
FKTNRKVTVKQLKEDYFKKIECFDSVEISGVEDRFNASLG
TYHDLLKlIKDKDFLDNEENEDILEDIVLTLTLFEDREMI
EERLKTYAHLFDDKVMKQLKRRRYTGWGRLSRKLINGIRD
KQSGKTILDFLKSDGFANRNFMQLIHDDSLTFKEDIQKAQ
VSGQGDSLHEHIANLAGSPAIKKGILQTVKVVDELVKVMG
RHKPENIVIEMARENQTTQKGQKNSRERMKRIEEGIKELG
SQILKEHPVENTQLQNEKLYLYYLQNGRDMYVDQELDINR
LSDYDVDAIVPQSFLKDDSIDNKVLTRSDKNRGKSDNVPS
EEVVKKMKNYWRQLLNAKLITQRKFDNLTKAERGGLSELD
KAGFIKRQLVETRQITKHVAQILDSRMNTKYDENDKLIRE
VKVITLKSKLVSDFRKDFQFYKVREINNYHHAHDAYLNAV
VGTALIKKYPKLESEFVYGDYKVYDVRKMIAKSEQEIGKA
TAKYFFYSNIMNFFKTEITLANGEIRKRPLIETNGETGEI
VWDKGRDFATVRKVLSMPQVNIVKKTEVQTGGFSKESILP
KRNSDKLIARKKDWDPKKYGGFDSPTVAYSVLVVAKVEKG
KSKKLKSVKELLGITIMERSSFEKNPIDFLEAKGYKEVKK
DLIIKLPKYSLFELENGRKRMLASAGELQKGNELALPSKY
VNFLYLASHYEKLKGSPEDNEQKQLFVEQHKHYLDEIIEQ
ISEFSKRVILADANLDKVLSAYNKHRDKPIREQAENIIHL
FTLTNLGAPAAFKYFDTTIDRKRYTSTKEVLDATLIHQSI
TGLYETRIDLSQLGGDPPKKKRKV
Seq ID NO 203:
>6his-NLS-A3A-GGS14-dCas9
HHHHHH-SSGLVPRGSHM-PKKKRKV-EASPASGPRHLMD
PHIFTSNFNNGIGRHKTYLCYEVERLDNGTSVKMDQHRGF
LHNQAKNLLCGFYGRHAELRFLDLVPSLQLDPAQIYRVTW
FISWSPCFSWGCAGEVRAFLQENTHVRLRIFAARIYDYDP
LYKEALQMLRDAGAQVSIMTYDEFKHCWDTFVDHQGCPFQ
PWDGLDEHSQALSGRLRAILQNQGN-GGSGGSGGSGGSGG
SGGSGGSGGSGGSGGSGGSGGSGGSGGS-MDKKYSIGLAI
GTNSVGWAVITDEYKVPSKKFKVLGNTDRHSIKKNLIGAL
LFDSGETAEATRLKRTARRRYTRRKNRICYLQEIFSNEMA
KVDDSFFHRLEESFLVEEDKKHERHPIFGNIVDEVAYHEK
YPTIYHLRKKLVDSTDKADLRLIYLALAHMIKFRGHFLIE
GDLNPDNSDVDKLFIQLVQTYNQLFEENPINASGVDAKAI
LSARLSKSRRLENLIAQLPGEKKNGLFGNLIALSLGLTPN
FKSNFDLAEDAKLQLSKDTYDDDLDNLLAQIGDQYADLFL
AAKNLSDAILLSDILRVNTEITKAPLSASMIKRYDEHHQD
LTLLKALVRQQLPEKYKEIFFDQSKNGYAGYIDGGASQEE
FYKFIKPILEKMDGTEELLVKLNREDLLRKQRTFDNGSIP
HQIHLGELHAILRRQEDFYPFLKDNREKIEKILTFRIPYY
VGPLARGNSRFAWMTRKSEETITPWNFEEVVDKGASAQSF
IERMTNFDKNLPNEKVLPKHSLLYEYFTVYNELTKVKYVT
EGMRKPAFLSGEQKKAIVDLLFKTNRKVTVKQLKEDYFKK
IECFDSVEISGVEDRFNASLGTYHDLLKlIKDKDFLDNEE
NEDILEDIVLTLTLFEDREMIEERLKTYAHLFDDKVMKQL
KRRRYTGWGRLSRKLINGIRDKQSGKTILDFLKSDGFANR
NFMQLIHDDSLTFKEDIQKAQVSGQGDSLHEHIANLAGSP
AIKKGILQTVKVVDELVKVMGRHKPENIVIEMARENQTTQ
KGQKNSRERMKRIEEGIKELGSQILKEHPVENTQLQNEKL
YLYYLQNGRDMYVDQELDINRLSDYDVDAIVPQSFLKDDS
IDNKVLTRSDKNRGKSDNVPSEEVVKKMKNYWRQLLNAKL
ITQRKFDNLTKAERGGLSELDKAGFIKRQLVETRQITKHV
AQILDSRMNTKYDENDKLIREVKVITLKSKLVSDFRKDFQ
FYKVREINNYHHAHDAYLNAVVGTALIKKYPKLESEFVYG
DYKVYDVRKMIAKSEQEIGKATAKYFFYSNIMNFFKTEIT
LANGEIRKRPLIETNGETGEIVWDKGRDFATVRKVLSMPQ
VNIVKKTEVQTGGFSKESILPKRNSDKLIARKKDWDPKKY
GGFDSPTVAYSVLVVAKVEKGKSKKLKSVKELLGITIMER
SSFEKNPIDFLEAKGYKEVKKDLIIKLPKYSLFELENGRK
RMLASAGELQKGNELALPSKYVNFLYLASHYEKLKGSPED
NEQKQLFVEQHKHYLDEIIEQISEFSKRVILADANLDKVL
SAYNKHRDKPIREQAENIIHLFTLTNLGAPAAFKYFDTTI
DRKRYTSTKEVLDATLIHQSITGLYETRIDLSQLGGDPPK
KKRKV
Seq ID NO 204:
>6his-NLS-A3H-GGS3-dCas9
HHHHHH-SSGLVPRGSHM-PKKKRKV-MALLTAETFRLQF
NNKRRLRRPYYPRKALLCYQLTPQNGSTPTRGYFENKKKC
HAEICFINEIKSMGLDETQCYQVTCYLTWSPCSSCAWELV
DFIKAHDHLNLRIFASRLYYHWCKPQQDGLRLLCGSQVPV
EVMGFPEFADCWENFVDHEKPLSFNPYKMLEELDKNSRAI
KRRLDRIKS-GGSGGSGGS-MDKKYSIGLAIGTNSVGWAV
ITDEYKVPSKKFKVLGNTDRHSIKKNLIGALLFDSGETAE
ATRLKRTARRRYTRRKNRICYLQEIFSNEMAKVDDSFFHR
LEESFLVEEDKKHERHPIFGNIVDEVAYHEKYPTIYHLRK
KLVDSTDKADLRLIYLALAHMIKFRGHFLIEGDLNPDNSD
VDKLFIQLVQTYNQLFEENPINASGVDAKAILSARLSKSR
RLENLIAQLPGEKKNGLFGNLIALSLGLTPNFKSNFDLAE
DAKLQLSKDTYDDDLDNLLAQIGDQYADLFLAAKNLSDAI
LLSDILRVNTEITKAPLSASMIKRYDEHHQDLTLLKALVR
QQLPEKYKEIFFDQSKNGYAGYIDGGASQEEFYKFIKPIL
EKMDGTEELLVKLNREDLLRKQRTFDNGSIPHQIHLGELH
AILRRQEDFYPFLKDNREKIEKILTFRIPYYVGPLARGNS
RFAWMTRKSEETITPWNFEEVVDKGASAQSFIERMTNFDK
NLPNEKVLPKHSLLYEYFTVYNELTKVKYVTEGMRKPAFL
SGEQKKAIVDLLFKTNRKVTVKQLKEDYFKKIECFDSVEI
SGVEDRFNASLGTYHDLLKIIKDKDFLDNEENEDILEDIV
LTLTLFEDREMIEERLKTYAHLFDDKVMKQLKRRRYTGWG
RLSRKLINGIRDKQSGKTILDFLKSDGFANRNFMQLIHDD
SLTFKEDIQKAQVSGQGDSLHEHIANLAGSPAIKKGILQT
VKVVDELVKVMGRHKPENIVIEMARENQTTQKGQKNSRER
MKRIEEGIKELGSQILKEHPVENTQLQNEKLYLYYLQNGR
DMYVDQELDINRLSDYDVDAIVPQSFLKDDSIDNKVLTRS
DKNRGKSDNVPSEEVVKKMKNYWRQLLNAKLITQRKFDNL
TKAERGGLSELDKAGFIKRQLVETRQITKHVAQILDSRMN
TKYDENDKLIREVKVITLKSKLVSDFRKDFQFYKVREINN
YHHAHDAYLNAVVGTALIKKYPKLESEFVYGDYKVYDVRK
MIAKSEQEIGKATAKYFFYSNIMNFFKTEITLANGEIRKR
PLIETNGETGEIVWDKGRDFATVRKVLSMPQVNIVKKTEV
QTGGFSKESILPKRNSDKLIARKKDWDPKKYGGFDSPTVA
YSVLVVAKVEKGKSKKLKSVKELLGITIMERSSFEKNPID
FLEAKGYKEVKKDLIKLPKYSLFELENGRKRMLASAGELQ
KGNELALPSKYVNFLYLASHYEKLKGSPEDNEQKQLFVEQ
HKHYLDEIIEQISEFSKRVILADANLDKVLSAYNKHRDKP
IREQAENIIHLFTLTNLGAPAAFKYFDTTIDRKRYTSTKE
VLDATLIHQSITGLYETRIDLSQLGGDPPKKKRKV
Seq ID NO 205:
>6his-NLS-A3H-GGS7-dCas9
HHHHHH-SSGLVPRGSHM-PKKKRKV-MALLTAETFRLQF
NNKRRLRRPYYPRKALLCYQLTPQNGSTPTRGYFENKKKC
HAEICFINEIKSMGLDETQCYQVTCYLTWSPCSSCAWELV
DFIKAHDHLNLRIFASRLYYHWCKPQQDGLRLLCGSQVPV
EVMGFPEFADCWENFVDHEKPLSFNPYKMLEELDKNSRAI
KRRLDRIKS-GGSGGSGGSGGSGGSGGSGGS-MDKKYSIG
LAIGTNSVGWAVITDEYKVPSKKFKVLGNTDRHSIKKNLI
GALLFDSGETAEATRLKRTARRRYTRRKNRICYLQEIFSN
EMAKVDDSFFHRLEESFLVEEDKKHERHPIFGNIVDEVAY
HEKYPTIYHLRKKLVDSTDKADLRLIYLALAHMIKFRGHF
LIEGDLNPDNSDVDKLFIQLVQTYNQLFEENPINASGVDA
KAILSARLSKSRRLENLIAQLPGEKKNGLFGNLIALSLGL
TPNFKSNFDLAEDAKLQLSKDTYDDDLDNLLAQIGDQYAD
LFLAAKNLSDAILLSDILRVNTEITKAPLSASMIKRYDEH
HQDLTLLKALVRQQLPEKYKEIFFDQSKNGYAGYIDGGAS
QEEFYKFIKPILEKMDGTEELLVKLNREDLLRKQRTFDNG
SIPHQIHLGELHAILRRQEDFYPFLKDNREKIEKILTFRI
PYYVGPLARGNSRFAWMTRKSEETITPWNFEEVVDKGASA
QSFIERMTNFDKNLPNEKVLPKHSLLYEYFTVYNELTKVK
YVTEGMRKPAFLSGEQKKAIVDLLFKTNRKVTVKQLKEDY
FKKIECFDSVEISGVEDRFNASLGTYHDLLKIIKDKDFLD
NEENEDILEDIVLTLTLFEDREMIEERLKTYAHLFDDKVM
KQLKRRRYTGWGRLSRKLINGIRDKQSGKTILDFLKSDGF
ANRNFMQLIHDDSLTFKEDIQKAQVSGQGDSLHEHIANLA
GSPAIKKGILQTVKVVDELVKVMGRHKPENIVIEMARENQ
TTQKGQKNSRERMKRIEEGIKELGSQILKEHPVENTQLQN
EKLYLYYLQNGRDMYVDQELDINRLSDYDVDAIVPQSFLK
DDSIDNKVLTRSDKNRGKSDNVPSEEVVKKMKNYWRQLLN
AKLITQRKFDNLTKAERGGLSELDKAGFIKRQLVETRQIT
KHVAQILDSRMNTKYDENDKLIREVKVITLKSKLVSDFRK
DFQFYKVREINNYHHAHDAYLNAVVGTALIKKYPKLESEF
VYGDYKVYDVRKMIAKSEQEIGKATAKYFFYSNIMNFFKT
EITLANGEIRKRPLIETNGETGEIVWDKGRDFATVRKVLS
MPQVNIVKKTEVQTGGFSKESILPKRNSDKLIARKKDWDP
KKYGGFDSPTVAYSVLVVAKVEKGKSKKLKSVKELLGITI
MERSSFEKNPIDFLEAKGYKEVKKDLIIKLPKYSLFELEN
GRKRMLASAGELQKGNELALPSKYVNFLYLASHYEKLKGS
PEDNEQKQLFVEQHKHYLDEIIEQISEFSKRVILADANLD
KVLSAYNKHRDKPIREQAENIIHLFTLTNLGAPAAFKYFD
TTIDRKRYTSTKEVLDATLIHQSITGLYETRIDLSQLGGD
PPKKKRKV
Seq ID NO 206:
>6his-NLS-A3H-GGS14-dCas9
HHHHHH-SSGLVPRGSHM-PKKKRKV-MALLTAETFRLQF
NNKRRLRRPYYPRKALLCYQLTPQNGSTPTRGYFENKKKC
HAEICFINEIKSMGLDETQCYQVTCYLTWSPCSSCAWELV
DFIKAHDHLNLRIFASRLYYHWCKPQQDGLRLLCGSQVPV
EVMGFPEFADCWENFVDHEKPLSFNPYKMLEELDKNSRAI
KRRLDRIKS-GGSGGSGGSGGSGGSGGSGGSGGSGGSGGS
GGSGGSGGSGGS-MDKKYSIGLAIGTNSVGWAVITDEYKV
PSKKFKVLGNTDRHSIKKNLIGALLFDSGETAEATRLKRT
ARRRYTRRKNRICYLQEIFSNEMAKVDDSFFHRLEESFLV
EEDKKHERHPIFGNIVDEVAYHEKYPTIYHLRKKLVDSTD
KADLRLIYLALAHMIKFRGHFLIEGDLNPDNSDVDKLFIQ
LVQTYNQLFEENPINASGVDAKAILSARLSKSRRLENLIA
QLPGEKKNGLFGNLIALSLGLTPNFKSNFDLAEDAKLQLS
KDTYDDDLDNLLAQIGDQYADLFLAAKNLSDAILLSDILR
VNTEITKAPLSASMIKRYDEHHQDLTLLKALVRQQLPEKY
KEIFFDQSKNGYAGYIDGGASQEEFYKFIKPILEKMDGTE
ELLVKLNREDLLRKQRTFDNGSIPHQIHLGELHAILRRQE
DFYPFLKDNREKIEKILTFRIPYYVGPLARGNSRFAWMTR
KSEETITPWNFEEVVDKGASAQSFIERMTNFDKNLPNEKV
LPKHSLLYEYFTVYNELTKVKYVTEGMRKPAFLSGEQKKA
IVDLLFKTNRKVTVKQLKEDYFKKIECFDSVEISGVEDRF
NASLGTYHDLLKlIKDKDFLDNEENEDILEDIVLTLTLFE
DREMIEERLKTYAHLFDDKVMKQLKRRRYTGWGRLSRKLI
NGIRDKQSGKTILDFLKSDGFANRNFMQLIHDDSLTFKED
IQKAQVSGQGDSLHEHIANLAGSPAIKKGILQTVKVVDEL
VKVMGRHKPENIVIEMARENQTTQKGQKNSRERMKRIEEG
IKELGSQILKEHPVENTQLQNEKLYLYYLQNGRDMYVDQE
LDINRLSDYDVDAIVPQSFLKDDSIDNKVLTRSDKNRGKS
DNVPSEEVVKKMKNYWRQLLNAKLITQRKFDNLTKAERGG
LSELDKAGFIKRQLVETRQITKHVAQILDSRMNTKYDEND
KLIREVKVITLKSKLVSDFRKDFQFYKVREINNYHHAHDA
YLNAVVGTALIKKYPKLESEFVYGDYKVYDVRKMIAKSEQ
EIGKATAKYFFYSNIMNFFKTEITLANGEIRKRPLIETNG
ETGEIVWDKGRDFATVRKVLSMPQVNIVKKTEVQTGGFSK
ESILPKRNSDKLIARKKDWDPKKYGGFDSPTVAYSVLVVA
KVEKGKSKKLKSVKELLGITIMERSSFEKNPIDFLEAKGY
KEVKKDLIIKLPKYSLFELENGRKRMLASAGELQKGNELA
LPSKYVNFLYLASHYEKLKGSPEDNEQKQLFVEQHKHYLD
EIIEQISEFSKRVILADANLDKVLSAYNKHRDKPIREQAE
NIIHLFTLTNLGAPAAFKYFDTTIDRKRYTSTKEVLDATL
IHQSITGLYETRIDLSQLGGDPPKKKRKV
Seq ID NO 207:
>6his-NLS-A3H-GGS7-dCpf1 gene sequence
HHHHHH-SSGLVPRGSHM-PKKKRKV-MALLTAETFRLQF
NNKRRLRRPYYPRKALLCYQLTPQNGSTPTRGYFENKKKC
HAEICFINEIKSMGLDETQCYQVTCYLTWSPCSSCAWELV
DFIKAHDHLNLRIFASRLYYHWCKPQQDGLRLLCGSQVPV
EVMGFPEFADCWENFVDHEKPLSFNPYKMLEELDKNSRAI
KRRLDRIKS-GGSGGSGGSGGSGGSGGSGGS-KLTQFEGF
TNLYQVSKTLRFELIPQGKTLKHIQEQGFIEEDKARNDHY
KELKPIIDRIYKTYADQCLQLVQLDWENLSAAIDSYRKEK
TEETRNALIEEQATYRNAIHDYFIGRTDNLTDAINKRHAE
IYKGLFKAELFNGKVLKQLGTVTTTEHENALLRSFDKFTT
YFSGFYENRKNVFSAEDISTAIPHRIVQDNFPKFKENCHI
FTRLITAVPSLREHFENVKKAIGIFVSTSIEEVFsFPFYN
QLLTQTQIDLYNQLLGGISREAGTEKIKGLNEVLNLAIQK
NDETAHIIASLPHRFIPLFKQILSDRNTLSFILEEFKSDE
EVIQSFCKYKTLLRNENVLETAEALFNELNSIDLTHIFIS
HKKLETISSALCDHWDTLRNALYERRISELTGKITKSAKE
KVQRSLKHEDINLQEIISAAGKELSEAFKQKTSEILSHAH
AALDQPLPTTLKKQEEKEILKSQLDSLLGLYHLLDWFAVD
ESNEVDPEFSARLTGIKLEMEPSLSFYNKARNYATKKPYS
VEKFKLNFQMPTLASGWDVNKEKNNGAILFVKNGLYYLGI
MPKQKGRYKALSFEPTEKTSEGFDKMYYDYFPDAAKMIPK
CSTQLKAVTAHFQTHTTPILLSNNFIEPLEITKEIYDLNN
PEKEPKKFQTAYAKKTGDQKGYREALCKWIDFTRDFLSKY
TKTTSIDLSSLRPSSQYKDLGEYYAELNPLLYHISFQRIA
EKEIMDAVETGKLYLFQIYNKDFAKGHHGKPNLHTLYWTG
LFSPENLAKTSIKLNGQAELFYRPKSRMKRMAHRLGEKML
NKKLKDQKTPIPDTLYQELYDYVNHRLSHDLSDEARALLP
NVITKEVSHEIIKDRRFTSDKFFFHVPITLNYQAANSPSK
FNQRVNAYLKEHPETPIIGIARGERNLIYITVIDSTGKIL
EQRSLNTIQQFDYQKKLDNREKERVAARQAWSVVGTIKDL
KQGYLSQVIHEIVDLMIHYQAVVVLENLNFGFKSKRTGIA
EKAVYQQFEKMLIDKLNCLVLKDYPAEKVGGVLNPYQLTD
QFTSFAKMGTQSGFLFYVPAPYTSKIDPLTGFVDPFVWKT
IKNHESRKHFLEGFDFLHYDVKTGDFILHFKMNRNLSFQR
GLPGFMPAWDIVFEKNETQFDAKGTPFIAGKRIVPVIENH
RFTGRYRDLYPANELIALLEEKGIVFRDGSNILPKLLEND
DSHAIDTMVALIRSVLQMRNSNAATGEDYINSPVRDLNGV
CFDSRFQNPEWPMDADANGAYHIALKGQLLLNHLKESKDL
KLQNGISNQDWLAYIQELRN
Seq ID NO 301:
>6his-NLS-A3A-GGS3-dCas9 gene sequence
ATGggcagcagccatcatcatcatcatcacagcagcggcc
tggtgccgcgcggcagccatatgccaaagaagaagcggaa
ggtcGAAGCCAGCCCAGCATCCGGGCCCAGACACTTGATG
GATCCACACATATTCACTTCCAACTTTAACAATGGCATTG
GAAGGCATAAGACCTACCTGTGCTACGAAGTGGAGCGCCT
GGACAATGGCACCTCGGTCAAGATGGACCAGCACAGGGGC
TTTCTACACAACCAGGCTAAGAATCTTCTCTGTGGCTTTT
ACGGCCGCCATGCGCAGCTGCGCTTCTTGGACCTGGTTCC
TTCTTTGCAGTTGGACCCGGCCCAGATCTACAGGGTCACT
TGGTTCATCTCCTGGAGCCCCTGCTTCTCCTGGGGCTGTG
CCGGGGAAGTGCGTGCGTTCCTTCAGGAGAACACACACGT
GAGACTGCGTATCTTCGCTGCCCGCATCTATGATTACGAC
CCCCTATATAAGGAGGCACTGCAAATGCTGCGGGATGCTG
GGGCCCAAGTCTCCATCATGACCTACGATGAATTTAAGCA
CTGCTGGGACACCTTTGTGGACCACCAGGGATGTCCCTTC
CAGCCCTGGGATGGACTAGATGAGCACAGCCAAGCCCTGA
GTGGGAGGCTGCGGGCCATTCTCCAGAATCAGGGAAACGG
AGGAAGTGGAGGAAGTGGAGGAAGTaagcttgacaagaag
tacagcatcggcctggccatcggcaccaactctgtgggct
gggccgtgatcaccgacgagtacaaggtgcccagcaagaa
attcaaggtgctgggcaacaccgaccggcacagcatcaag
aagaacctgatcggagccctgctgttcgacagcggcgaaa
cagccgaggccacccggctgaagagaaccgccagaagaag
atacaccagacggaagaaccggatctgctatctgcaagag
atcttcagcaacgagatggccaaggtggacgacagcttct
tccacagactggaagagtccttcctggtggaagaggataa
gaagcacgagcggcaccccatcttcggcaacatcgtggac
gaggtggcctaccacgagaagtaccccaccatctaccacc
tgagaaagaaactggtggacagcaccgacaaggccgacct
gcggctgatctatctggccctggcccacatgatcaagttc
cggggccacttcctgatcgagggcgacctgaaccccgaca
acagcgacgtggacaagctgttcatccagctggtgcagac
ctacaaccagctgttcgaggaaaaccccatcaacgccagc
ggcgtggacgccaaggccatcctgtctgccagactgagca
agagcagacggctggaaaatctgatcgcccagctgcccgg
cgagaagaagaatggcctgttcggaaacctgattgccctg
agcctgggcctgacccccaacttcaagagcaacttcgacc
tggccgaggatgccaaactgcagctgagcaaggacaccta
cgacgacgacctggacaacctgctggcccagatcggcgac
cagtacgccgacctgtttctggccgccaagaacctgtccg
acgccatcctgctgagcgacatcctgagagtgaacaccga
gatcaccaaggcccccctgagcgcctctatgatcaagaga
tacgacgagcaccaccaggacctgaccctgctgaaagctc
tcgtgcggcagcagctgcctgagaagtacaaagagatttt
cttcgaccagagcaagaacggctacgccggctacattgac
ggcggagccagccaggaagagttctacaagttcatcaagc
ccatcctggaaaagatggacggcaccgaggaactgctcgt
gaagctgaacagagaggacctgctgcggaagcagcggacc
ttcgacaacggcagcatcccccaccagatccacctgggag
agctgcacgccattctgcggcggcaggaagatttttaccc
attcctgaaggacaaccgggaaaagatcgagaagatcctg
accttccgcatcccctactacgtgggccctctggccaggg
gaaacagcagattcgcctggatgaccagaaagagcgagga
aaccatcaccccctggaacttcgaggaagtggtggacaag
ggcgcttccgcccagagcttcatcgagcggatgaccaact
tcgataagaacctgcccaacgagaaggtgctgcccaagca
cagcctgctgtacgagtacttcaccgtgtataacgagctg
accaaagtgaaatacgtgaccgagggaatgagaaagcccg
ccttcctgagcggcgagcagaaaaaggccatcgtggacct
gctgttcaagaccaaccggaaagtgaccgtgaagcagctg
aaagaggactacttcaagaaaatcgagtgcttcgactccg
tggaaatctccggcgtggaagatcggttcaacgcctccct
gggcacataccacgatctgctgaaaattatcaaggacaag
gacttcctggacaatgaggaaaacgaggacattctggaag
atatcgtgctgaccctgacactgtttgaggacagagagat
gatcgaggaacggctgaaaacctatgcccacctgttcgac
gacaaagtgatgaagcagctgaagcggcggagatacaccg
gctggggcaggctgagccggaagctgatcaacggcatccg
ggacaagcagtccggcaagacaatcctggatttcctgaag
tccgacggcttcgccaacagaaacttcatgcagctgatcc
acgacgacagcctgacctttaaagaggacatccagaaagc
ccaggtgtccggccagggcgatagcctgcacgagcacatt
gccaatctggccggcagccccgccattaagaagggcatcc
tgcagacagtgaaggtggtggacgagctcgtgaaagtgat
gggccggcacaagcccgagaacatcgtgatcgaaatggcc
agagagaaccagaccacccagaagggacagaagaacagcc
gcgagagaatgaagcggatcgaagagggcatcaaagagct
gggcagccagatcctgaaagaacaccccgtggaaaacacc
cagctgcagaacgagaagctgtacctgtactacctgcaga
atgggcgggatatgtacgtggaccaggaactggacatcaa
ccggctgtccgactacgatgtggacgctatcgtgcctcag
agctttctgaaggacgactccatcgacaacaaggtgctga
ccagaagcgacaagaaccggggcaagagcgacaacgtgcc
ctccgaagaggtcgtgaagaagatgaagaactactggcgg
cagctgctgaacgccaagctgattacccagagaaagttcg
acaatctgaccaaggccgagagaggcggcctgagcgaact
ggataaggccggcttcatcaagagacagctggtggaaacc
cggcagatcacaaagcacgtggcacagatcctggactccc
ggatgaacactaagtacgacgagaatgacaagctgatccg
ggaagtgaaagtgatcaccctgaagtccaagctggtgtcc
gatttccggaaggatttccagttttacaaagtgcgcgaga
tcaacaactaccaccacgcccacgacgcctacctgaacgc
cgtcgtgggaaccgccctgatcaaaaagtaccctaagctg
gaaagcgagttcgtgtacggcgactacaaggtgtacgacg
tgcggaagatgatcgccaagagcgagcaggaaatcggcaa
ggctaccgccaagtacttcttctacagcaacatcatgaac
tttttcaagaccgagattaccctggccaacggcgagatcc
ggaagcggcctctgatcgagacaaacggcgaaaccgggga
gatcgtgtgggataagggccgggattttgccaccgtgcgg
aaagtgctgagcatgccccaagtgaatatcgtgaaaaaga
ccgaggtgcagacaggcggcttcagcaaagagtctatcct
gcccaagaggaacagcgataagctgatcgccagaaagaag
gactgggaccctaagaagtacggcggcttcgacagcccca
ccgtggcctattctgtgctggtggtggccaaagtggaaaa
gggcaagtccaagaaactgaagagtgtgaaagagctgctg
gggatcaccatcatggaaagaagcagcttcgagaagaatc
ccatcgactttctggaagccaagggctacaaagaagtgaa
aaaggacctgatcatcaagctgcctaagtactccctgttc
gagctggaaaacggccggaagagaatgctggcctctgccg
gcgaactgcagaagggaaacgaactggccctgccctccaa
atatgtgaacttcctgtacctggccagccactatgagaag
ctgaagggctcccccgaggataatgagcagaaacagctgt
ttgtggaacagcacaagcactacctggacgagatcatcga
gcagatcagcgagttctccaagagagtgatcctggccgac
gctaatctggacaaagtgctgtccgcctacaacaagcacc
gggataagcccatcagagagcaggccgagaatatcatcca
cctgtttaccctgaccaatctgggagcccctgccgccttc
aagtactttgacaccaccatcgaccggaagaggtacacca
gcaccaaagaggtgctggacgccaccctgatccaccagag
catcaccggcctgtacgagacacggatcgacctgtctcag
ctgggaggcgactaactcgag
Seq ID NO 302:
>6his-NLS-A3A-GGS7-dCas9 gene sequence
ATGggcagcagccatcatcatcatcatcacagcagcggcc
tggtgccgcgcggcagccatatgccaaagaagaagcggaa
ggtcGAAGCCAGCCCAGCATCCGGGCCCAGACACTTGATG
GATCCACACATATTCACTTCCAACTTTAACAATGGCATTG
GAAGGCATAAGACCTACCTGTGCTACGAAGTGGAGCGCCT
GGACAATGGCACCTCGGTCAAGATGGACCAGCACAGGGGC
TTTCTACACAACCAGGCTAAGAATCTTCTCTGTGGCTTTT
ACGGCCGCCATGCGCAGCTGCGCTTCTTGGACCTGGTTCC
TTCTTTGCAGTTGGACCCGGCCCAGATCTACAGGGTCACT
TGGTTCATCTCCTGGAGCCCCTGCTTCTCCTGGGGCTGTG
CCGGGGAAGTGCGTGCGTTCCTTCAGGAGAACACACACGT
GAGACTGCGTATCTTCGCTGCCCGCATCTATGATTACGAC
CCCCTATATAAGGAGGCACTGCAAATGCTGCGGGATGCTG
GGGCCCAAGTCTCCATCATGACCTACGATGAATTTAAGCA
CTGCTGGGACACCTTTGTGGACCACCAGGGATGTCCCTTC
CAGCCCTGGGATGGACTAGATGAGCACAGCCAAGCCCTGA
GTGGGAGGCTGCGGGCCATTCTCCAGAATCAGGGAAACGG
AGGAAGTGGAGGAAGTGGAGGAAGTGGAGGAAGTGGAGGA
AGTGGAGGAAGTGGAGGAAGTaagcttgacaagaagtaca
gcatcggcctggccatcggcaccaactctgtgggctgggc
cgtgatcaccgacgagtacaaggtgcccagcaagaaattc
aaggtgctgggcaacaccgaccggcacagcatcaagaaga
acctgatcggagccctgctgttcgacagcggcgaaacagc
cgaggccacccggctgaagagaaccgccagaagaagatac
accagacggaagaaccggatctgctatctgcaagagatct
tcagcaacgagatggccaaggtggacgacagcttcttcca
cagactggaagagtccttcctggtggaagaggataagaag
cacgagcggcaccccatcttcggcaacatcgtggacgagg
tggcctaccacgagaagtaccccaccatctaccacctgag
aaagaaactggtggacagcaccgacaaggccgacctgcgg
ctgatctatctggccctggcccacatgatcaagttccggg
gccacttcctgatcgagggcgacctgaaccccgacaacag
cgacgtggacaagctgttcatccagctggtgcagacctac
aaccagctgttcgaggaaaaccccatcaacgccagcggcg
tggacgccaaggccatcctgtctgccagactgagcaagag
cagacggctggaaaatctgatcgcccagctgcccggcgag
aagaagaatggcctgttcggaaacctgattgccctgagcc
tgggcctgacccccaacttcaagagcaacttcgacctggc
cgaggatgccaaactgcagctgagcaaggacacctacgac
gacgacctggacaacctgctggcccagatcggcgaccagt
acgccgacctgtttctggccgccaagaacctgtccgacgc
catcctgctgagcgacatcctgagagtgaacaccgagatc
accaaggcccccctgagcgcctctatgatcaagagatacg
acgagcaccaccaggacctgaccctgctgaaagctctcgt
gcggcagcagctgcctgagaagtacaaagagattttcttc
gaccagagcaagaacggctacgccggctacattgacggcg
gagccagccaggaagagttctacaagttcatcaagcccat
cctggaaaagatggacggcaccgaggaactgctcgtgaag
ctgaacagagaggacctgctgcggaagcagcggaccttcg
acaacggcagcatcccccaccagatccacctgggagagct
gcacgccattctgcggcggcaggaagatttttacccattc
ctgaaggacaaccgggaaaagatcgagaagatcctgacct
tccgcatcccctactacgtgggccctctggccaggggaaa
cagcagattcgcctggatgaccagaaagagcgaggaaacc
atcaccccctggaacttcgaggaagtggtggacaagggcg
cttccgcccagagcttcatcgagcggatgaccaacttcga
taagaacctgcccaacgagaaggtgctgcccaagcacagc
ctgctgtacgagtacttcaccgtgtataacgagctgacca
aagtgaaatacgtgaccgagggaatgagaaagcccgcctt
cctgagcggcgagcagaaaaaggccatcgtggacctgctg
ttcaagaccaaccggaaagtgaccgtgaagcagctgaaag
aggactacttcaagaaaatcgagtgcttcgactccgtgga
aatctccggcgtggaagatcggttcaacgcctccctgggc
acataccacgatctgctgaaaattatcaaggacaaggact
tcctggacaatgaggaaaacgaggacattctggaagatat
cgtgctgaccctgacactgtttgaggacagagagatgatc
gaggaacggctgaaaacctatgcccacctgttcgacgaca
aagtgatgaagcagctgaagcggcggagatacaccggctg
gggcaggctgagccggaagctgatcaacggcatccgggac
aagcagtccggcaagacaatcctggatttcctgaagtccg
acggcttcgccaacagaaacttcatgcagctgatccacga
cgacagcctgacctttaaagaggacatccagaaagcccag
gtgtccggccagggcgatagcctgcacgagcacattgcca
atctggccggcagccccgccattaagaagggcatcctgca
gacagtgaaggtggtggacgagctcgtgaaagtgatgggc
cggcacaagcccgagaacatcgtgatcgaaatggccagag
agaaccagaccacccagaagggacagaagaacagccgcga
gagaatgaagcggatcgaagagggcatcaaagagctgggc
agccagatcctgaaagaacaccccgtggaaaacacccagc
tgcagaacgagaagctgtacctgtactacctgcagaatgg
gcgggatatgtacgtggaccaggaactggacatcaaccgg
ctgtccgactacgatgtggacgctatcgtgcctcagagct
ttctgaaggacgactccatcgacaacaaggtgctgaccag
aagcgacaagaaccggggcaagagcgacaacgtgccctcc
gaagaggtcgtgaagaagatgaagaactactggcggcagc
tgctgaacgccaagctgattacccagagaaagttcgacaa
tctgaccaaggccgagagaggcggcctgagcgaactggat
aaggccggcttcatcaagagacagctggtggaaacccggc
agatcacaaagcacgtggcacagatcctggactcccggat
gaacactaagtacgacgagaatgacaagctgatccgggaa
gtgaaagtgatcaccctgaagtccaagctggtgtccgatt
tccggaaggatttccagttttacaaagtgcgcgagatcaa
caactaccaccacgcccacgacgcctacctgaacgccgtc
gtgggaaccgccctgatcaaaaagtaccctaagctggaaa
gcgagttcgtgtacggcgactacaaggtgtacgacgtgcg
gaagatgatcgccaagagcgagcaggaaatcggcaaggct
accgccaagtacttcttctacagcaacatcatgaactttt
tcaagaccgagattaccctggccaacggcgagatccggaa
gcggcctctgatcgagacaaacggcgaaaccggggagatc
gtgtgggataagggccgggattttgccaccgtgcggaaag
tgctgagcatgccccaagtgaatatcgtgaaaaagaccga
ggtgcagacaggcggcttcagcaaagagtctatcctgccc
aagaggaacagcgataagctgatcgccagaaagaaggact
gggaccctaagaagtacggcggcttcgacagccccaccgt
ggcctattctgtgctggtggtggccaaagtggaaaagggc
aagtccaagaaactgaagagtgtgaaagagctgctgggga
tcaccatcatggaaagaagcagcttcgagaagaatcccat
cgactttctggaagccaagggctacaaagaagtgaaaaag
gacctgatcatcaagctgcctaagtactccctgttcgagc
tggaaaacggccggaagagaatgctggcctctgccggcga
actgcagaagggaaacgaactggccctgccctccaaatat
gtgaacttcctgtacctggccagccactatgagaagctga
agggctcccccgaggataatgagcagaaacagctgtttgt
ggaacagcacaagcactacctggacgagatcatcgagcag
atcagcgagttctccaagagagtgatcctggccgacgcta
atctggacaaagtgctgtccgcctacaacaagcaccggga
taagcccatcagagagcaggccgagaatatcatccacctg
tttaccctgaccaatctgggagcccctgccgccttcaagt
actttgacaccaccatcgaccggaagaggtacaccagcac
caaagaggtgctggacgccaccctgatccaccagagcatc
accggcctgtacgagacacggatcgacctgtctcagctgg
gaggcgactaactcgag
Seq ID NO 303:
>6his-NLS-A3A-GGS14-dCas9 gene sequence
ATGggcagcagccatcatcatcatcatcacagcagcggcc
tggtgccgcgcggcagccatatgccaaagaagaagcggaa
ggtcGAAGCCAGCCCAGCATCCGGGCCCAGACACTTGATG
GATCCACACATATTCACTTCCAACTTTAACAATGGCATTG
GAAGGCATAAGACCTACCTGTGCTACGAAGTGGAGCGCCT
GGACAATGGCACCTCGGTCAAGATGGACCAGCACAGGGGC
TTTCTACACAACCAGGCTAAGAATCTTCTCTGTGGCTTTT
ACGGCCGCCATGCGCAGCTGCGCTTCTTGGACCTGGTTCC
TTCTTTGCAGTTGGACCCGGCCCAGATCTACAGGGTCACT
TGGTTCATCTCCTGGAGCCCCTGCTTCTCCTGGGGCTGTG
CCGGGGAAGTGCGTGCGTTCCTTCAGGAGAACACACACGT
GAGACTGCGTATCTTCGCTGCCCGCATCTATGATTACGAC
CCCCTATATAAGGAGGCACTGCAAATGCTGCGGGATGCTG
GGGCCCAAGTCTCCATCATGACCTACGATGAATTTAAGCA
CTGCTGGGACACCTTTGTGGACCACCAGGGATGTCCCTTC
CAGCCCTGGGATGGACTAGATGAGCACAGCCAAGCCCTGA
GTGGGAGGCTGCGGGCCATTCTCCAGAATCAGGGAAACGG
AGGAAGTGGAGGAAGTGGAGGAAGTGGAGGAAGTGGAGGA
AGTGGAGGAAGTGGAGGAAGTGGAGGAAGTGGAGGAAGTG
GAGGAAGTGGAGGAAGTGGAGGAAGTGGAGGAAGTGGAGG
AAGTaagcttgacaagaagtacagcatcggcctggccatc
ggcaccaactctgtgggctgggccgtgatcaccgacgagt
acaaggtgcccagcaagaaattcaaggtgctgggcaacac
cgaccggcacagcatcaagaagaacctgatcggagccctg
ctgttcgacagcggcgaaacagccgaggccacccggctga
agagaaccgccagaagaagatacaccagacggaagaaccg
gatctgctatctgcaagagatcttcagcaacgagatggcc
aaggtggacgacagcttcttccacagactggaagagtcct
tcctggtggaagaggataagaagcacgagcggcaccccat
cttcggcaacatcgtggacgaggtggcctaccacgagaag
taccccaccatctaccacctgagaaagaaactggtggaca
gcaccgacaaggccgacctgcggctgatctatctggccct
ggcccacatgatcaagttccggggccacttcctgatcgag
ggcgacctgaaccccgacaacagcgacgtggacaagctgt
tcatccagctggtgcagacctacaaccagctgttcgagga
aaaccccatcaacgccagcggcgtggacgccaaggccatc
ctgtctgccagactgagcaagagcagacggctggaaaatc
tgatcgcccagctgcccggcgagaagaagaatggcctgtt
cggaaacctgattgccctgagcctgggcctgacccccaac
ttcaagagcaacttcgacctggccgaggatgccaaactgc
agctgagcaaggacacctacgacgacgacctggacaacct
gctggcccagatcggcgaccagtacgccgacctgtttctg
gccgccaagaacctgtccgacgccatcctgctgagcgaca
tcctgagagtgaacaccgagatcaccaaggcccccctgag
cgcctctatgatcaagagatacgacgagcaccaccaggac
ctgaccctgctgaaagctctcgtgcggcagcagctgcctg
agaagtacaaagagattttcttcgaccagagcaagaacgg
ctacgccggctacattgacggcggagccagccaggaagag
ttctacaagttcatcaagcccatcctggaaaagatggacg
gcaccgaggaactgctcgtgaagctgaacagagaggacct
gctgcggaagcagcggaccttcgacaacggcagcatcccc
caccagatccacctgggagagctgcacgccattctgcggc
ggcaggaagatttttacccattcctgaaggacaaccggga
aaagatcgagaagatcctgaccttccgcatcccctactac
gtgggccctctggccaggggaaacagcagattcgcctgga
tgaccagaaagagcgaggaaaccatcaccccctggaactt
cgaggaagtggtggacaagggcgcttccgcccagagcttc
atcgagcggatgaccaacttcgataagaacctgcccaacg
agaaggtgctgcccaagcacagcctgctgtacgagtactt
caccgtgtataacgagctgaccaaagtgaaatacgtgacc
gagggaatgagaaagcccgccttcctgagcggcgagcaga
aaaaggccatcgtggacctgctgttcaagaccaaccggaa
agtgaccgtgaagcagctgaaagaggactacttcaagaaa
atcgagtgcttcgactccgtggaaatctccggcgtggaag
atcggttcaacgcctccctgggcacataccacgatctgct
gaaaattatcaaggacaaggacttcctggacaatgaggaa
aacgaggacattctggaagatatcgtgctgaccctgacac
tgtttgaggacagagagatgatcgaggaacggctgaaaac
ctatgcccacctgttcgacgacaaagtgatgaagcagctg
aagcggcggagatacaccggctggggcaggctgagccgga
agctgatcaacggcatccgggacaagcagtccggcaagac
aatcctggatttcctgaagtccgacggcttcgccaacaga
aacttcatgcagctgatccacgacgacagcctgaccttta
aagaggacatccagaaagcccaggtgtccggccagggcga
tagcctgcacgagcacattgccaatctggccggcagcccc
gccattaagaagggcatcctgcagacagtgaaggtggtgg
acgagctcgtgaaagtgatgggccggcacaagcccgagaa
catcgtgatcgaaatggccagagagaaccagaccacccag
aagggacagaagaacagccgcgagagaatgaagcggatcg
aagagggcatcaaagagctgggcagccagatcctgaaaga
acaccccgtggaaaacacccagctgcagaacgagaagctg
tacctgtactacctgcagaatgggcgggatatgtacgtgg
accaggaactggacatcaaccggctgtccgactacgatgt
ggacgctatcgtgcctcagagctttctgaaggacgactcc
atcgacaacaaggtgctgaccagaagcgacaagaaccggg
gcaagagcgacaacgtgccctccgaagaggtcgtgaagaa
gatgaagaactactggcggcagctgctgaacgccaagctg
attacccagagaaagttcgacaatctgaccaaggccgaga
gaggcggcctgagcgaactggataaggccggcttcatcaa
gagacagctggtggaaacccggcagatcacaaagcacgtg
gcacagatcctggactcccggatgaacactaagtacgacg
agaatgacaagctgatccgggaagtgaaagtgatcaccct
gaagtccaagctggtgtccgatttccggaaggatttccag
ttttacaaagtgcgcgagatcaacaactaccaccacgccc
acgacgcctacctgaacgccgtcgtgggaaccgccctgat
caaaaagtaccctaagctggaaagcgagttcgtgtacggc
gactacaaggtgtacgacgtgcggaagatgatcgccaaga
gcgagcaggaaatcggcaaggctaccgccaagtacttctt
ctacagcaacatcatgaactttttcaagaccgagattacc
ctggccaacggcgagatccggaagcggcctctgatcgaga
caaacggcgaaaccggggagatcgtgtgggataagggccg
ggattttgccaccgtgcggaaagtgctgagcatgccccaa
gtgaatatcgtgaaaaagaccgaggtgcagacaggcggct
tcagcaaagagtctatcctgcccaagaggaacagcgataa
gctgatcgccagaaagaaggactgggaccctaagaagtac
ggcggcttcgacagccccaccgtggcctattctgtgctgg
tggtggccaaagtggaaaagggcaagtccaagaaactgaa
gagtgtgaaagagctgctggggatcaccatcatggaaaga
agcagcttcgagaagaatcccatcgactttctggaagcca
agggctacaaagaagtgaaaaaggacctgatcatcaagct
gcctaagtactccctgttcgagctggaaaacggccggaag
agaatgctggcctctgccggcgaactgcagaagggaaacg
aactggccctgccctccaaatatgtgaacttcctgtacct
ggccagccactatgagaagctgaagggctcccccgaggat
aatgagcagaaacagctgtttgtggaacagcacaagcact
acctggacgagatcatcgagcagatcagcgagttctccaa
gagagtgatcctggccgacgctaatctggacaaagtgctg
tccgcctacaacaagcaccgggataagcccatcagagagc
aggccgagaatatcatccacctgtttaccctgaccaatct
gggagcccctgccgccttcaagtactttgacaccaccatc
gaccggaagaggtacaccagcaccaaagaggtgctggacg
ccaccctgatccaccagagcatcaccggcctgtacgagac
acggatcgacctgtctcagctgggaggcgactaactcgag
Seq ID NO 304:
>6his-NLS-A3H-GGS3-dCas9 gene sequence
ATGggcagcagccatcatcatcatcatcacagcagcggcc
tggtgccgcgcggcagccatatgccaaagaagaagcggaa
ggtcGCTCTTCTTACTGCTGAAACTTTTCGTCTCCAATTT
AATAATAAACGCCGTCTGCGTCGCCCGTATTACCCGCGCA
AGGCGCTGCTGTGTTACCAACTGACCCCACAAAACGGTTC
CACCCCGACTCGCGGTTACTTTGAGAATAAGAAAAAATGT
CACGCTGAGATCTGTTTCATTAACGAAATCAAATCTATGG
GCCTGGATGAAACTCAGTGCTACCAGGTCACCTGCTACCT
GACCTGGAGCCCGTGTAGCTCTTGCGCGTGGGAACTGGTT
GACTTCATCAAAGCGCACGACCATCTGAACCTGCGTATCT
TCGCTTCCCGCCTGTACTATCACTGGTGCAAGCCGCAACA
GGATGGCCTGCGCCTGCTGTGTGGTTCTCAGGTTCCGGTT
GAAGTTATGGGTTTCCCGGAGTTTGCGGACTGCTGGGAAA
ACTTTGTTGACCATGAGAAGCCACTGTCCTTTAACCCGTA
TAAAATGCTGGAAGAGCTGGACAAAAACTCTCGTGCTATC
AAGCGCCGTCTGGATCGTATCAAGTCTGGAGGAAGTGGAG
GAAGTGGAGGAAGTagcttgacaagaagtacagcatcggc
ctggccatcggcaccaactctgtgggctgggccgtgatca
ccgacgagtacaaggtgcccagcaagaaattcaaggtgct
gggcaacaccgaccggcacagcatcaagaagaacctgatc
ggagccctgctgttcgacagcggcgaaacagccgaggcca
cccggctgaagagaaccgccagaagaagatacaccagacg
gaagaaccggatctgctatctgcaagagatcttcagcaac
gagatggccaaggtggacgacagcttcttccacagactgg
aagagtccttcctggtggaagaggataagaagcacgagcg
gcaccccatcttcggcaacatcgtggacgaggtggcctac
cacgagaagtaccccaccatctaccacctgagaaagaaac
tggtggacagcaccgacaaggccgacctgcggctgatcta
tctggccctggcccacatgatcaagttccggggccacttc
ctgatcgagggcgacctgaaccccgacaacagcgacgtgg
acaagctgttcatccagctggtgcagacctacaaccagct
gttcgaggaaaaccccatcaacgccagcggcgtggacgcc
aaggccatcctgtctgccagactgagcaagagcagacggc
tggaaaatctgatcgcccagctgcccggcgagaagaagaa
tggcctgttcggaaacctgattgccctgagcctgggcctg
acccccaacttcaagagcaacttcgacctggccgaggatg
ccaaactgcagctgagcaaggacacctacgacgacgacct
ggacaacctgctggcccagatcggcgaccagtacgccgac
ctgtttctggccgccaagaacctgtccgacgccatcctgc
tgagcgacatcctgagagtgaacaccgagatcaccaaggc
ccccctgagcgcctctatgatcaagagatacgacgagcac
caccaggacctgaccctgctgaaagctctcgtgcggcagc
agctgcctgagaagtacaaagagattttcttcgaccagag
caagaacggctacgccggctacattgacggcggagccagc
caggaagagttctacaagttcatcaagcccatcctggaaa
agatggacggcaccgaggaactgctcgtgaagctgaacag
agaggacctgctgcggaagcagcggaccttcgacaacggc
agcatcccccaccagatccacctgggagagctgcacgcca
ttctgcggcggcaggaagatttttacccattcctgaagga
caaccgggaaaagatcgagaagatcctgaccttccgcatc
ccctactacgtgggccctctggccaggggaaacagcagat
tcgcctggatgaccagaaagagcgaggaaaccatcacccc
ctggaacttcgaggaagtggtggacaagggcgcttccgcc
cagagcttcatcgagcggatgaccaacttcgataagaacc
tgcccaacgagaaggtgctgcccaagcacagcctgctgta
cgagtacttcaccgtgtataacgagctgaccaaagtgaaa
tacgtgaccgagggaatgagaaagcccgccttcctgagcg
gcgagcagaaaaaggccatcgtggacctgctgttcaagac
caaccggaaagtgaccgtgaagcagctgaaagaggactac
ttcaagaaaatcgagtgcttcgactccgtggaaatctccg
gcgtggaagatcggttcaacgcctccctgggcacatacca
cgatctgctgaaaattatcaaggacaaggacttcctggac
aatgaggaaaacgaggacattctggaagatatcgtgctga
ccctgacactgtttgaggacagagagatgatcgaggaacg
gctgaaaacctatgcccacctgttcgacgacaaagtgatg
aagcagctgaagcggcggagatacaccggctggggcaggc
tgagccggaagctgatcaacggcatccgggacaagcagtc
cggcaagacaatcctggatttcctgaagtccgacggcttc
gccaacagaaacttcatgcagctgatccacgacgacagcc
tgacctttaaagaggacatccagaaagcccaggtgtccgg
ccagggcgatagcctgcacgagcacattgccaatctggcc
ggcagccccgccattaagaagggcatcctgcagacagtga
aggtggtggacgagctcgtgaaagtgatgggccggcacaa
gcccgagaacatcgtgatcgaaatggccagagagaaccag
accacccagaagggacagaagaacagccgcgagagaatga
agcggatcgaagagggcatcaaagagctgggcagccagat
cctgaaagaacaccccgtggaaaacacccagctgcagaac
gagaagctgtacctgtactacctgcagaatgggcgggata
tgtacgtggaccaggaactggacatcaaccggctgtccga
ctacgatgtggacgctatcgtgcctcagagctttctgaag
gacgactccatcgacaacaaggtgctgaccagaagcgaca
agaaccggggcaagagcgacaacgtgccctccgaagaggt
cgtgaagaagatgaagaactactggcggcagctgctgaac
gccaagctgattacccagagaaagttcgacaatctgacca
aggccgagagaggcggcctgagcgaactggataaggccgg
cttcatcaagagacagctggtggaaacccggcagatcaca
aagcacgtggcacagatcctggactcccggatgaacacta
agtacgacgagaatgacaagctgatccgggaagtgaaagt
gatcaccctgaagtccaagctggtgtccgatttccggaag
gatttccagttttacaaagtgcgcgagatcaacaactacc
accacgcccacgacgcctacctgaacgccgtcgtgggaac
cgccctgatcaaaaagtaccctaagctggaaagcgagttc
gtgtacggcgactacaaggtgtacgacgtgcggaagatga
tcgccaagagcgagcaggaaatcggcaaggctaccgccaa
gtacttcttctacagcaacatcatgaactttttcaagacc
gagattaccctggccaacggcgagatccggaagcggcctc
tgatcgagacaaacggcgaaaccggggagatcgtgtggga
taagggccgggattttgccaccgtgcggaaagtgctgagc
atgccccaagtgaatatcgtgaaaaagaccgaggtgcaga
caggcggcttcagcaaagagtctatcctgcccaagaggaa
cagcgataagctgatcgccagaaagaaggactgggaccct
aagaagtacggcggcttcgacagccccaccgtggcctatt
ctgtgctggtggtggccaaagtggaaaagggcaagtccaa
gaaactgaagagtgtgaaagagctgctggggatcaccatc
atggaaagaagcagcttcgagaagaatcccatcgactttc
tggaagccaagggctacaaagaagtgaaaaaggacctgat
catcaagctgcctaagtactccctgttcgagctggaaaac
ggccggaagagaatgctggcctctgccggcgaactgcaga
agggaaacgaactggccctgccctccaaatatgtgaactt
cctgtacctggccagccactatgagaagctgaagggctcc
cccgaggataatgagcagaaacagctgtttgtggaacagc
acaagcactacctggacgagatcatcgagcagatcagcga
gttctccaagagagtgatcctggccgacgctaatctggac
aaagtgctgtccgcctacaacaagcaccgggataagccca
tcagagagcaggccgagaatatcatccacctgtttaccct
gaccaatctgggagcccctgccgccttcaagtactttgac
accaccatcgaccggaagaggtacaccagcaccaaagagg
tgctggacgccaccctgatccaccagagcatcaccggcct
gtacgagacacggatcgacctgtctcagctgggaggcgac
taactcgag
Seq ID NO 305:
>6his-NLS-A3H-GGS7-dCas9 gene sequence
ATGggcagcagccatcatcatcatcatcacagcagcggcc
tggtgccgcgcggcagccatatgccaaagaagaagcggaa
ggtcGCTCTTCTTACTGCTGAAACTTTTCGTCTCCAATTT
AATAATAAACGCCGTCTGCGTCGCCCGTATTACCCGCGCA
AGGCGCTGCTGTGTTACCAACTGACCCCACAAAACGGTTC
CACCCCGACTCGCGGTTACTTTGAGAATAAGAAAAAATGT
CACGCTGAGATCTGTTTCATTAACGAAATCAAATCTATGG
GCCTGGATGAAACTCAGTGCTACCAGGTCACCTGCTACCT
GACCTGGAGCCCGTGTAGCTCTTGCGCGTGGGAACTGGTT
GACTTCATCAAAGCGCACGACCATCTGAACCTGCGTATCT
TCGCTTCCCGCCTGTACTATCACTGGTGCAAGCCGCAACA
GGATGGCCTGCGCCTGCTGTGTGGTTCTCAGGTTCCGGTT
GAAGTTATGGGTTTCCCGGAGTTTGCGGACTGCTGGGAAA
ACTTTGTTGACCATGAGAAGCCACTGTCCTTTAACCCGTA
TAAAATGCTGGAAGAGCTGGACAAAAACTCTCGTGCTATC
AAGCGCCGTCTGGATCGTATCAAGTCTGGAGGAAGTGGAG
GAAGTGGAGGAAGTGGAGGAAGTGGAGGAAGTGGAGGAAG
TGGAGGAAGTaagcttgacaagaagtacagcatcggcctg
gccatcggcaccaactctgtgggctgggccgtgatcaccg
acgagtacaaggtgcccagcaagaaattcaaggtgctggg
caacaccgaccggcacagcatcaagaagaacctgatcgga
gccctgctgttcgacagcggcgaaacagccgaggccaccc
ggctgaagagaaccgccagaagaagatacaccagacggaa
gaaccggatctgctatctgcaagagatcttcagcaacgag
atggccaaggtggacgacagcttcttccacagactggaag
agtccttcctggtggaagaggataagaagcacgagcggca
ccccatcttcggcaacatcgtggacgaggtggcctaccac
gagaagtaccccaccatctaccacctgagaaagaaactgg
tggacagcaccgacaaggccgacctgcggctgatctatct
ggccctggcccacatgatcaagttccggggccacttcctg
atcgagggcgacctgaaccccgacaacagcgacgtggaca
agctgttcatccagctggtgcagacctacaaccagctgtt
cgaggaaaaccccatcaacgccagcggcgtggacgccaag
gccatcctgtctgccagactgagcaagagcagacggctgg
aaaatctgatcgcccagctgcccggcgagaagaagaatgg
cctgttcggaaacctgattgccctgagcctgggcctgacc
cccaacttcaagagcaacttcgacctggccgaggatgcca
aactgcagctgagcaaggacacctacgacgacgacctgga
caacctgctggcccagatcggcgaccagtacgccgacctg
tttctggccgccaagaacctgtccgacgccatcctgctga
gcgacatcctgagagtgaacaccgagatcaccaaggcccc
cctgagcgcctctatgatcaagagatacgacgagcaccac
caggacctgaccctgctgaaagctctcgtgcggcagcagc
tgcctgagaagtacaaagagattttcttcgaccagagcaa
gaacggctacgccggctacattgacggcggagccagccag
gaagagttctacaagttcatcaagcccatcctggaaaaga
tggacggcaccgaggaactgctcgtgaagctgaacagaga
ggacctgctgcggaagcagcggaccttcgacaacggcagc
atcccccaccagatccacctgggagagctgcacgccattc
tgcggcggcaggaagatttttacccattcctgaaggacaa
ccgggaaaagatcgagaagatcctgaccttccgcatcccc
tactacgtgggccctctggccaggggaaacagcagattcg
cctggatgaccagaaagagcgaggaaaccatcaccccctg
gaacttcgaggaagtggtggacaagggcgcttccgcccag
agcttcatcgagcggatgaccaacttcgataagaacctgc
ccaacgagaaggtgctgcccaagcacagcctgctgtacga
gtacttcaccgtgtataacgagctgaccaaagtgaaatac
gtgaccgagggaatgagaaagcccgccttcctgagcggcg
agcagaaaaaggccatcgtggacctgctgttcaagaccaa
ccggaaagtgaccgtgaagcagctgaaagaggactacttc
aagaaaatcgagtgcttcgactccgtggaaatctccggcg
tggaagatcggttcaacgcctccctgggcacataccacga
tctgctgaaaattatcaaggacaaggacttcctggacaat
gaggaaaacgaggacattctggaagatatcgtgctgaccc
tgacactgtttgaggacagagagatgatcgaggaacggct
gaaaacctatgcccacctgttcgacgacaaagtgatgaag
cagctgaagcggcggagatacaccggctggggcaggctga
gccggaagctgatcaacggcatccgggacaagcagtccgg
caagacaatcctggatttcctgaagtccgacggcttcgcc
aacagaaacttcatgcagctgatccacgacgacagcctga
cctttaaagaggacatccagaaagcccaggtgtccggcca
gggcgatagcctgcacgagcacattgccaatctggccggc
agccccgccattaagaagggcatcctgcagacagtgaagg
tggtggacgagctcgtgaaagtgatgggccggcacaagcc
cgagaacatcgtgatcgaaatggccagagagaaccagacc
acccagaagggacagaagaacagccgcgagagaatgaagc
ggatcgaagagggcatcaaagagctgggcagccagatcct
gaaagaacaccccgtggaaaacacccagctgcagaacgag
aagctgtacctgtactacctgcagaatgggcgggatatgt
acgtggaccaggaactggacatcaaccggctgtccgacta
cgatgtggacgctatcgtgcctcagagctttctgaaggac
gactccatcgacaacaaggtgctgaccagaagcgacaaga
accggggcaagagcgacaacgtgccctccgaagaggtcgt
gaagaagatgaagaactactggcggcagctgctgaacgcc
aagctgattacccagagaaagttcgacaatctgaccaagg
ccgagagaggcggcctgagcgaactggataaggccggctt
catcaagagacagctggtggaaacccggcagatcacaaag
cacgtggcacagatcctggactcccggatgaacactaagt
acgacgagaatgacaagctgatccgggaagtgaaagtgat
caccctgaagtccaagctggtgtccgatttccggaaggat
ttccagttttacaaagtgcgcgagatcaacaactaccacc
acgcccacgacgcctacctgaacgccgtcgtgggaaccgc
cctgatcaaaaagtaccctaagctggaaagcgagttcgtg
tacggcgactacaaggtgtacgacgtgcggaagatgatcg
ccaagagcgagcaggaaatcggcaaggctaccgccaagta
cttcttctacagcaacatcatgaactttttcaagaccgag
attaccctggccaacggcgagatccggaagcggcctctga
tcgagacaaacggcgaaaccggggagatcgtgtgggataa
gggccgggattttgccaccgtgcggaaagtgctgagcatg
ccccaagtgaatatcgtgaaaaagaccgaggtgcagacag
gcggcttcagcaaagagtctatcctgcccaagaggaacag
cgataagctgatcgccagaaagaaggactgggaccctaag
aagtacggcggcttcgacagccccaccgtggcctattctg
tgctggtggtggccaaagtggaaaagggcaagtccaagaa
actgaagagtgtgaaagagctgctggggatcaccatcatg
gaaagaagcagcttcgagaagaatcccatcgactttctgg
aagccaagggctacaaagaagtgaaaaaggacctgatcat
caagctgcctaagtactccctgttcgagctggaaaacggc
cggaagagaatgctggcctctgccggcgaactgcagaagg
gaaacgaactggccctgccctccaaatatgtgaacttcct
gtacctggccagccactatgagaagctgaagggctccccc
gaggataatgagcagaaacagctgtttgtggaacagcaca
agcactacctggacgagatcatcgagcagatcagcgagtt
ctccaagagagtgatcctggccgacgctaatctggacaaa
gtgctgtccgcctacaacaagcaccgggataagcccatca
gagagcaggccgagaatatcatccacctgtttaccctgac
caatctgggagcccctgccgccttcaagtactttgacacc
accatcgaccggaagaggtacaccagcaccaaagaggtgc
tggacgccaccctgatccaccagagcatcaccggcctgta
cgagacacggatcgacctgtctcagctgggaggcgactaa
ctcgag
Seq ID NO 306:
>6his-NLS-A3H-GGS14-dCas9 gene sequence
ATGggcagcagccatcatcatcatcatcacagcagcggcc
tggtgccgcgcggcagccatatgccaaagaagaagcggaa
ggtcGCTCTTCTTACTGCTGAAACTTTTCGTCTCCAATTT
AATAATAAACGCCGTCTGCGTCGCCCGTATTACCCGCGCA
AGGCGCTGCTGTGTTACCAACTGACCCCACAAAACGGTTC
CACCCCGACTCGCGGTTACTTTGAGAATAAGAAAAAATGT
CACGCTGAGATCTGTTTCATTAACGAAATCAAATCTATGG
GCCTGGATGAAACTCAGTGCTACCAGGTCACCTGCTACCT
GACCTGGAGCCCGTGTAGCTCTTGCGCGTGGGAACTGGTT
GACTTCATCAAAGCGCACGACCATCTGAACCTGCGTATCT
TCGCTTCCCGCCTGTACTATCACTGGTGCAAGCCGCAACA
GGATGGCCTGCGCCTGCTGTGTGGTTCTCAGGTTCCGGTT
GAAGTTATGGGTTTCCCGGAGTTTGCGGACTGCTGGGAAA
ACTTTGTTGACCATGAGAAGCCACTGTCCTTTAACCCGTA
TAAAATGCTGGAAGAGCTGGACAAAAACTCTCGTGCTATC
AAGCGCCGTCTGGATCGTATCAAGTCTGGAGGAAGTGGAG
GAAGTGGAGGAAGTGGAGGAAGTGGAGGAAGTGGAGGAAG
TGGAGGAAGTGGAGGAAGTGGAGGAAGTGGAGGAAGTGGA
GGAAGTGGAGGAAGTGGAGGAAGTGGAGGAAGTaagcttg
acaagaagtacagcatcggcctggccatcggcaccaactc
tgtgggctgggccgtgatcaccgacgagtacaaggtgccc
agcaagaaattcaaggtgctgggcaacaccgaccggcaca
gcatcaagaagaacctgatcggagccctgctgttcgacag
cggcgaaacagccgaggccacccggctgaagagaaccgcc
agaagaagatacaccagacggaagaaccggatctgctatc
tgcaagagatcttcagcaacgagatggccaaggtggacga
cagcttcttccacagactggaagagtccttcctggtggaa
gaggataagaagcacgagcggcaccccatcttcggcaaca
tcgtggacgaggtggcctaccacgagaagtaccccaccat
ctaccacctgagaaagaaactggtggacagcaccgacaag
gccgacctgcggctgatctatctggccctggcccacatga
tcaagttccggggccacttcctgatcgagggcgacctgaa
ccccgacaacagcgacgtggacaagctgttcatccagctg
gtgcagacctacaaccagctgttcgaggaaaaccccatca
acgccagcggcgtggacgccaaggccatcctgtctgccag
actgagcaagagcagacggctggaaaatctgatcgcccag
ctgcccggcgagaagaagaatggcctgttcggaaacctga
ttgccctgagcctgggcctgacccccaacttcaagagcaa
cttcgacctggccgaggatgccaaactgcagctgagcaag
gacacctacgacgacgacctggacaacctgctggcccaga
tcggcgaccagtacgccgacctgtttctggccgccaagaa
cctgtccgacgccatcctgctgagcgacatcctgagagtg
aacaccgagatcaccaaggcccccctgagcgcctctatga
tcaagagatacgacgagcaccaccaggacctgaccctgct
gaaagctctcgtgcggcagcagctgcctgagaagtacaaa
gagattttcttcgaccagagcaagaacggctacgccggct
acattgacggcggagccagccaggaagagttctacaagtt
catcaagcccatcctggaaaagatggacggcaccgaggaa
ctgctcgtgaagctgaacagagaggacctgctgcggaagc
agcggaccttcgacaacggcagcatcccccaccagatcca
cctgggagagctgcacgccattctgcggcggcaggaagat
ttttacccattcctgaaggacaaccgggaaaagatcgaga
agatcctgaccttccgcatcccctactacgtgggccctct
ggccaggggaaacagcagattcgcctggatgaccagaaag
agcgaggaaaccatcaccccctggaacttcgaggaagtgg
tggacaagggcgcttccgcccagagcttcatcgagcggat
gaccaacttcgataagaacctgcccaacgagaaggtgctg
cccaagcacagcctgctgtacgagtacttcaccgtgtata
acgagctgaccaaagtgaaatacgtgaccgagggaatgag
aaagcccgccttcctgagcggcgagcagaaaaaggccatc
gtggacctgctgttcaagaccaaccggaaagtgaccgtga
agcagctgaaagaggactacttcaagaaaatcgagtgctt
cgactccgtggaaatctccggcgtggaagatcggttcaac
gcctccctgggcacataccacgatctgctgaaaattatca
aggacaaggacttcctggacaatgaggaaaacgaggacat
tctggaagatatcgtgctgaccctgacactgtttgaggac
agagagatgatcgaggaacggctgaaaacctatgcccacc
tgttcgacgacaaagtgatgaagcagctgaagcggcggag
atacaccggctggggcaggctgagccggaagctgatcaac
ggcatccgggacaagcagtccggcaagacaatcctggatt
tcctgaagtccgacggcttcgccaacagaaacttcatgca
gctgatccacgacgacagcctgacctttaaagaggacatc
cagaaagcccaggtgtccggccagggcgatagcctgcacg
agcacattgccaatctggccggcagccccgccattaagaa
gggcatcctgcagacagtgaaggtggtggacgagctcgtg
aaagtgatgggccggcacaagcccgagaacatcgtgatcg
aaatggccagagagaaccagaccacccagaagggacagaa
gaacagccgcgagagaatgaagcggatcgaagagggcatc
aaagagctgggcagccagatcctgaaagaacaccccgtgg
aaaacacccagctgcagaacgagaagctgtacctgtacta
cctgcagaatgggcgggatatgtacgtggaccaggaactg
gacatcaaccggctgtccgactacgatgtggacgctatcg
tgcctcagagctttctgaaggacgactccatcgacaacaa
ggtgctgaccagaagcgacaagaaccggggcaagagcgac
aacgtgccctccgaagaggtcgtgaagaagatgaagaact
actggcggcagctgctgaacgccaagctgattacccagag
aaagttcgacaatctgaccaaggccgagagaggcggcctg
agcgaactggataaggccggcttcatcaagagacagctgg
tggaaacccggcagatcacaaagcacgtggcacagatcct
ggactcccggatgaacactaagtacgacgagaatgacaag
ctgatccgggaagtgaaagtgatcaccctgaagtccaagc
tggtgtccgatttccggaaggatttccagttttacaaagt
gcgcgagatcaacaactaccaccacgcccacgacgcctac
ctgaacgccgtcgtgggaaccgccctgatcaaaaagtacc
ctaagctggaaagcgagttcgtgtacggcgactacaaggt
gtacgacgtgcggaagatgatcgccaagagcgagcaggaa
atcggcaaggctaccgccaagtacttcttctacagcaaca
tcatgaactttttcaagaccgagattaccctggccaacgg
cgagatccggaagcggcctctgatcgagacaaacggcgaa
accggggagatcgtgtgggataagggccgggattttgcca
ccgtgcggaaagtgctgagcatgccccaagtgaatatcgt
gaaaaagaccgaggtgcagacaggcggcttcagcaaagag
tctatcctgcccaagaggaacagcgataagctgatcgcca
gaaagaaggactgggaccctaagaagtacggcggcttcga
cagccccaccgtggcctattctgtgctggtggtggccaaa
gtggaaaagggcaagtccaagaaactgaagagtgtgaaag
agctgctggggatcaccatcatggaaagaagcagcttcga
gaagaatcccatcgactttctggaagccaagggctacaaa
gaagtgaaaaaggacctgatcatcaagctgcctaagtact
ccctgttcgagctggaaaacggccggaagagaatgctggc
ctctgccggcgaactgcagaagggaaacgaactggccctg
ccctccaaatatgtgaacttcctgtacctggccagccact
atgagaagctgaagggctcccccgaggataatgagcagaa
acagctgtttgtggaacagcacaagcactacctggacgag
atcatcgagcagatcagcgagttctccaagagagtgatcc
tggccgacgctaatctggacaaagtgctgtccgcctacaa
caagcaccgggataagcccatcagagagcaggccgagaat
atcatccacctgtttaccctgaccaatctgggagcccctg
ccgccttcaagtactttgacaccaccatcgaccggaagag
gtacaccagcaccaaagaggtgctggacgccaccctgatc
caccagagcatcaccggcctgtacgagacacggatcgacc
tgtctcagctgggaggcgactaactcgag
Seq ID NO 307:
>6his-NLS-A3H-GGS7-dCpf1 gene sequence
ATGggcagcagccatcatcatcatcatcacagcagcggcc
tggtgccgcgcggcagccatatgccaaagaagaagcggaa
ggtcGCTCTTCTTACTGCTGAAACTTTTCGTCTCCAATTT
AATAATAAACGCCGTCTGCGTCGCCCGTATTACCCGCGCA
AGGCGCTGCTGTGTTACCAACTGACCCCACAAAACGGTTC
CACCCCGACTCGCGGTTACTTTGAGAATAAGAAAAAATGT
CACGCTGAGATCTGTTTCATTAACGAAATCAAATCTATGG
GCCTGGATGAAACTCAGTGCTACCAGGTCACCTGCTACCT
GACCTGGAGCCCGTGTAGCTCTTGCGCGTGGGAACTGGTT
GACTTCATCAAAGCGCACGACCATCTGAACCTGCGTATCT
TCGCTTCCCGCCTGTACTATCACTGGTGCAAGCCGCAACA
GGATGGCCTGCGCCTGCTGTGTGGTTCTCAGGTTCCGGTT
GAAGTTATGGGTTTCCCGGAGTTTGCGGACTGCTGGGAAA
ACTTTGTTGACCATGAGAAGCCACTGTCCTTTAACCCGTA
TAAAATGCTGGAAGAGCTGGACAAAAACTCTCGTGCTATC
AAGCGCCGTCTGGATCGTATCAAGTCTGGAGGAAGTGGAG
GAAGTGGAGGAAGTGGAGGAAGTGGAGGAAGTGGAGGAAG
TGGAGGAAGTATGACACAGTTCGAGGGCTTTACCAACCTG
TATCAGGTGAGCAAGACACTGCGGTTTGAGCTGATCCCAC
AGGGCAAGACCCTGAAGCACATCCAGGAGCAGGGCTTCAT
CGAGGAGGACAAGGCCCGCAATGATCACTACAAGGAGCTG
AAGCCCATCATCGATCGGATCTACAAGACCTATGCCGACC
AGTGCCTGCAGCTGGTGCAGCTGGATTGGGAGAACCTGAG
CGCCGCCATCGACTCCTATAGAAAGGAGAAAACCGAGGAG
ACAAGGAACGCCCTGATCGAGGAGCAGGCCACATATCGCA
ATGCCATCCACGACTACTTCATCGGCCGGACAGACAACCT
GACCGATGCCATCAATAAGAGACACGCCGAGATCTACAAG
GGCCTGTTCAAGGCCGAGCTGTTTAATGGCAAGGTGCTGA
AGCAGCTGGGCACCGTGACCACAACCGAGCACGAGAACGC
CCTGCTGCGGAGCTTCGACAAGTTTACAACCTACTTCTCC
GGCTTTTATGAGAACAGGAAGAACGTGTTCAGCGCCGAGG
ATATCAGCACAGCCATCCCACACCGCATCGTGCAGGACAA
CTTCCCCAAGTTTAAGGAGAATTGTCACATCTTCACACGC
CTGATCACCGCCGTGCCCAGCCTGCGGGAGCACTTTGAGA
ACGTGAAGAAGGCCATCGGCATCTTCGTGAGCACCTCCAT
CGAGGAGGTGTTTTCCTTCCCTTTTTATAACCAGCTGCTG
ACACAGACCCAGATCGACCTGTATAACCAGCTGCTGGGAG
GAATCTCTCGGGAGGCAGGCACCGAGAAGATCAAGGGCCT
GAACGAGGTGCTGAATCTGGCCATCCAGAAGAATGATGAG
ACAGCCCACATCATCGCCTCCCTGCCACACAGATTCATCC
CCCTGTTTAAGCAGATCCTGTCCGATAGGAACACCCTGTC
TTTCATCCTGGAGGAGTTTAAGAGCGACGAGGAAGTGATC
CAGTCCTTCTGCAAGTACAAGACACTGCTGAGAAACGAGA
ACGTGCTGGAGACAGCCGAGGCCCTGTTTAACGAGCTGAA
CAGCATCGACCTGACACACATCTTCATCAGCCACAAGAAG
CTGGAGACAATCAGCAGCGCCCTGTGCGACCACTGGGATA
CACTGAGGAATGCCCTGTATGAGCGGAGAATCTCCGAGCT
GACAGGCAAGATCACCAAGTCTGCCAAGGAGAAGGTGCAG
CGCAGCCTGAAGCACGAGGATATCAACCTGCAGGAGATCA
TCTCTGCCGCAGGCAAGGAGCTGAGCGAGGCCTTCAAGCA
GAAAACCAGCGAGATCCTGTCCCACGCACACGCCGCCCTG
GATCAGCCACTGCCTACAACCCTGAAGAAGCAGGAGGAGA
AGGAGATCCTGAAGTCTCAGCTGGACAGCCTGCTGGGCCT
GTACCACCTGCTGGACTGGTTTGCCGTGGATGAGTCCAAC
GAGGTGGACCCCGAGTTCTCTGCCCGGCTGACCGGCATCA
AGCTGGAGATGGAGCCTTCTCTGAGCTTCTACAACAAGGC
CAGAAATTATGCCACCAAGAAGCCCTACTCCGTGGAGAAG
TTCAAGCTGAACTTTCAGATGCCTACACTGGCCTCTGGCT
GGGACGTGAATAAGGAGAAGAACAATGGCGCCATCCTGTT
TGTGAAGAACGGCCTGTACTATCTGGGCATCATGCCAAAG
CAGAAGGGCAGGTATAAGGCCCTGAGCTTCGAGCCCACAG
AGAAAACCAGCGAGGGCTTTGATAAGATGTACTATGACTA
CTTCCCTGATGCCGCCAAGATGATCCCAAAGTGCAGCACC
CAGCTGAAGGCCGTGACAGCCCACTTTCAGACCCACACAA
CCCCCATCCTGCTGTCCAACAATTTCATCGAGCCTCTGGA
GATCACAAAGGAGATCTACGACCTGAACAATCCTGAGAAG
GAGCCAAAGAAGTTTCAGACAGCCTACGCCAAGAAAACCG
GCGACCAGAAGGGCTACAGAGAGGCCCTGTGCAAGTGGAT
CGACTTCACAAGGGATTTTCTGTCCAAGTATACCAAGACA
ACCTCTATCGATCTGTCTAGCCTGCGGCCATCCTCTCAGT
ATAAGGACCTGGGCGAGTACTATGCCGAGCTGAATCCCCT
GCTGTACCACATCAGCTTCCAGAGAATCGCCGAGAAGGAG
ATCATGGATGCCGTGGAGACAGGCAAGCTGTACCTGTTCC
AGATCTATAACAAGGACTTTGCCAAGGGCCACCACGGCAA
GCCTAATCTGCACACACTGTATTGGACCGGCCTGTTTTCT
CCAGAGAACCTGGCCAAGACAAGCATCAAGCTGAATGGCC
AGGCCGAGCTGTTCTACCGCCCTAAGTCCAGGATGAAGAG
GATGGCACACCGGCTGGGAGAGAAGATGCTGAACAAGAAG
CTGAAGGATCAGAAAACCCCAATCCCCGACACCCTGTACC
AGGAGCTGTACGACTATGTGAATCACAGACTGTCCCACGA
CCTGTCTGATGAGGCCAGGGCCCTGCTGCCCAACGTGATC
ACCAAGGAGGTGTCTCACGAGATCATCAAGGATAGGCGCT
TTACCAGCGACAAGTTCTTTTTCCACGTGCCTATCACACT
GAACTATCAGGCCGCCAATTCCCCATCTAAGTTCAACCAG
AGGGTGAATGCCTACCTGAAGGAGCACCCCGAGACACCTA
TCATCGGCATCGATCGGGGCGAGAGAAACCTGATCTATAT
CACAGTGATCGACTCCACCGGCAAGATCCTGGAGCAGCGG
AGCCTGAACACCATCCAGCAGTTTGATTACCAGAAGAAGC
TGGACAACAGGGAGAAGGAGAGGGTGGCAGCAAGGCAGGC
CTGGTCTGTGGTGGGCACAATCAAGGATCTGAAGCAGGGC
TATCTGAGCCAGGTCATCCACGAGATCGTGGACCTGATGA
TCCACTACCAGGCCGTGGTGGTGCTGGAGAACCTGAATTT
CGGCTTTAAGAGCAAGAGGACCGGCATCGCCGAGAAGGCC
GTGTACCAGCAGTTCGAGAAGATGCTGATCGATAAGCTGA
ATTGCCTGGTGCTGAAGGACTATCCAGCAGAGAAAGTGGG
AGGCGTGCTGAACCCATACCAGCTGACAGACCAGTTCACC
TCCTTTGCCAAGATGGGCACCCAGTCTGGCTTCCTGTTTT
ACGTGCCTGCCCCATATACATCTAAGATCGATCCCCTGAC
CGGCTTCGTGGACCCCTTCGTGTGGAAAACCATCAAGAAT
CACGAGAGCCGCAAGCACTTCCTGGAGGGCTTCGACTTTC
TGCACTACGACGTGAAAACCGGCGACTTCATCCTGCACTT
TAAGATGAACAGAAATCTGTCCTTCCAGAGGGGCCTGCCC
GGCTTTATGCCTGCATGGGATATCGTGTTCGAGAAGAACG
AGACACAGTTTGACGCCAAGGGCACCCCTTTCATCGCCGG
CAAGAGAATCGTGCCAGTGATCGAGAATCACAGATTCACC
GGCAGATACCGGGACCTGTATCCTGCCAACGAGCTGATCG
CCCTGCTGGAGGAGAAGGGCATCGTGTTCAGGGATGGCTC
CAACATCCTGCCAAAGCTGCTGGAGAATGACGATTCTCAC
GCCATCGACACCATGGTGGCCCTGATCCGCAGCGTGCTGC
AGATGCGGAACTCCAATGCCGCCACAGGCGAGGACTATAT
CAACAGCCCCGTGCGCGATCTGAATGGCGTGTGCTTCGAC
TCCCGGTTTCAGAACCCAGAGTGGCCCATGGACGCCGATG
CCAATGGCGCCTACCACATCGCCCTGAAGGGCCAGCTGCT
GCTGAATCACCTGAAGGAGAGCAAGGATCTGAAGCTGCAG
AACGGCATCTCCAATCAGGACTGGCTGGCCTACATCCAGG
AGCTGCGCAACAAAAGGCCGGCGGCCACGAAAAAGGCCGG
CCAGGCAAAAAAGAAAAAGGGATCCTACCCATACGATGTT
CCAGATTACGCTTATCCCTACGACGTGCCTGATTATGCAT
ACCCATATGATGTCCCCGACTATGCCTAAG

1. A method for editing a target nucleic acid molecule, comprising the steps of:
obtaining a recombinant vector encoding a fusion protein and a small guide RNA (sgRNA), wherein the fusion protein comprises an Apobec family protein domain at N-terminal and a Cas9 family or a Cpf1 family protein domain whose nuclease activity is inactivated at C-terminal, and the small guide RNA has a complementary region to a target editing region of the target nucleic acid molecule, wherein the target editing region of the target nucleic acid molecule includes at least one methylated cytosine nucleotide;
contacting the recombinant vector encoding the fusion protein and the small guide RNA (sgRNA) obtained in the step with the target nucleic acid molecule.

2. The method for editing a target nucleic acid molecule according to claim 1, wherein the Apobec family protein at N-terminal of the fusion protein is selected from the group consisting of human Apobec3A or Apobec3H, or a protein having deamination activity with 95% or more homology to human Apobec3A or Apobec3H.

3. The method for editing a target nucleic acid molecule according to claim 1, wherein the protein sequence of the Cas9 protein whose nuclease activity is inactivated at C-terminal of the fusion protein is a mutant sequence in which aspartic acid at position 10 and histidine at position 840 are mutated to alanine and alanine, the protein sequence of the Cpf1 protein whose nuclease activity is inactivated at C-terminal of the fusion protein is a mutant sequence in which aspartic acid is mutated to alanine at position 908.

4. The method for editing a target nucleic acid molecule according to claim 1, wherein between the two domains of the fusion protein is a linker consisting of 3-14 motifs.

5. The method for editing a target nucleic acid molecule according to claim 4, wherein the motif is selected from (GGS).

6. The method for editing a target nucleic acid molecule according to claim 1, wherein the fusion protein further comprises a purification tag sequence.

7. The method for editing a target nucleic acid molecule according to claim 1, wherein the fusion protein is selected from any of SEQ ID NOs. 201-207.

8. A gene sequence encoding the protein sequence of claim 7.

9. (canceled)

10. The method for editing a target nucleic acid molecule according to claim 1, wherein the small guide RNA is 60-80 bp in length.

11. The method for editing a target nucleic acid molecule according to claim 1, wherein a complementary region of the small guide RNA to the target nucleic acid molecule is 18-25 bp in length.

12. A method for editing a target nucleic acid molecule in vitro, comprising the steps of:
obtaining a recombinant vector encoding a fusion protein and a small guide RNA (sgRNA), the fusion protein comprises an Apobec family protein domain at N-terminal and a Cas9 family or a Cpf1 family protein domain whose nuclease activity is inactivated at C-terminal, and the small guide RNA has a complementary region to a target editing region of the target nucleic acid molecule, wherein the target editing region of the target nucleic acid molecule includes at least one methylated cytosine nucleotide;
contacting the fusion protein and the small guide RNA (sgRNA) with the target nucleic acid molecule;
after a high temperature termination reaction, adding an effective amount of TDG and carring out a reaction at 42° C. for 6 to 8 hours; and
adding an effective amount of EDTA, formamide and NaOH, and carrying out a reaction at 90 to 95° C. for 5 to 10 minutes.

13. The method for editing a target nucleic acid molecule according to claim 1, wherein the methylated cytidine nucleotide is associated with diseases such as cancer, genetic disorders, developmental errors and the like.

14.-15. (canceled)

16. The method for editing a target nucleic acid molecule according to claim 12, wherein the methylated cytidine nucleotide is associated with diseases such as cancer, genetic disorders, developmental errors and the like.
